(19) United States
(12) Patent Application Publication
(10) Pub. No.: US 2026/0062394 A1
(43) Pub. Date: Mar. 5, 2026

(54) PROCESSES FOR PREPARING OXAZOLIDINONE COMPOUNDS

(71) Applicants: Merck Sharp & Dohme LLC, Rahway, NJ (US); Werthenstein BioPharma GmbH, Schachen (CH)

(72) Inventors: David Harris, New Providence, NJ (US); Patrick Larpent, Lucerne (CH); Zhao Liu, Closter, NJ (US); Ashish Punia, Westfield, NJ (US); Wes A. Schafer, New York, NY (US); Lushi Tan, Edison, NJ (US); Yingju Xu, Westfield, NJ (US); Chintal Desai, Flemington, NJ (US)

(73) Assignees: Merck Sharp & Dohme LLC, Rahway, NJ (US); Werthenstein BioPharma GmbH, Schachen (CH)

(21) Appl. No.: 19/101,012

(22) PCT Filed: Aug. 7, 2023
(86) PCT No.: PCT/US2023/029590

§ 371 Date: Feb. 4, 2025

Related U.S. Application Data
(60) Provisional application No. 63/396,829, filed on Aug. 10, 2022.

(51) Int. Cl. C07D 263/20; A61K 9/16; A61K 31/541; C07C 271/16; C07D 295/135

(52) U.S. Cl. CPC C07D 263/20; A61K 9/1635; A61K 9/1652; A61K 31/541; C07C 271/16; C07D 295/135

(57) ABSTRACT

The invention relates to crystalline forms and processes for preparing a compound of Formula I (I) and pharmaceutically acceptable salts thereof which are useful for the treatment of bacterial infections, particularly mycobacterial infections. The invention also relates to intermediates used in the processes for preparing the compound of Formula I for the treatment of mycobacterial infections such as those caused by Mycobacteria tuberculosis.

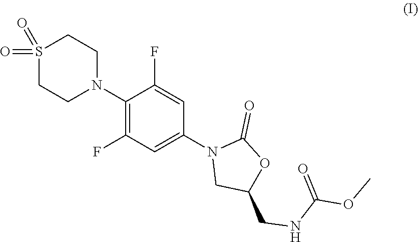 [SMILES: COC(=O)NC[C@H]1CN(c2cc(F)c(N3CCS(=O)(=O)CC3)c(F)c2)C(=O)O1]

### CROSS-REFERENCE TO RELATED APPLICATIONS

This application claims the benefit of U.S. Provisional Patent Application Ser. No. 63/396,829 filed Aug. 10, 2022, the entire contents of which are incorporated by reference herein.

### FIELD OF THE INVENTION

The invention relates to crystalline forms of and processes for preparing oxazolidinone compounds useful for the treatment of bacterial infections, particularly mycobacterial infections. More specifically, the invention relates to novel crystalline forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, useful for the treatment and prevention of mycobacterial infections such as those caused by Mycobacteria tuberculosis. The invention also relates to processes for preparing and intermediates used in the processes for preparing crystalline forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, useful for the treatment and prevention of mycobacterial infections such as those caused by Mycobacteria tuberculosis. The invention further relates to pharmaceutical compositions of crystalline and amorphous forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

### BACKGROUND OF THE INVENTION

Mycobacterium is a genus of bacterium, neither truly gram-positive nor truly gram-negative, including pathogens responsible for tuberculosis (M. tuberculosis) and leprosy (M. leprae). Tuberculosis (TB), in particular, despite the availability of anti-TB drugs such as isoniazid and rifampin, is considered to be one of the world's deadliest diseases. According to World Health Organization, in 2018, there were 10 million new TB cases and 1.5 million TB deaths. See, Global Tuberculosis Report 2019 published by the World Health Organization. Complicating the TB epidemic is the rising tide of multi-drug-resistant strains, and the deadly association with HIV. People who are HIV-positive and infected with TB are 30 times more likely to develop active TB than people who are HIV-negative, and TB is responsible for the death of one out of every three people with HIV/AIDS worldwide. See, e.g, Kaufmann et al., Trends Microbiol. 1: 2-5 (1993) and Bloom et al., N Engl. J Med. 338: 677-678 (1998).

Mycobacteria other than M. tuberculosis are increasingly found in opportunistic infections that plague the AIDS patient. Organisms from the M. avium-intracellular complex (MAC), especially serotypes four and eight, account for 68% of the mycobacterial isolates from AIDS patients. Enormous numbers of MAC are found (up to 1010 acid-fast bacilli per gram of tissue), and consequently, the prognosis for the infected AIDS patient is poor.

Oxazolidinones are a class of compounds containing 2-oxazolidone, a 5-membered ring containing nitrogen and oxygen, which are used as antimicrobials. See, e.g, WO 2009157423. In general, oxazolidinones are known to be monoamine oxidase inhibitors and to have activity against gram-positive microorganisms. WO 2006022794, Suzuki et al., Med. Chem. Lett. 4:1074-1078 (2013), Yang et al., J. Med. Chem. 58:6389-6409 (2015), Shaw et al., Ann. N.Y. Acad. Sci. 1241:48-70 (2011). Additionally, PCT Publication No. WO2017/070024 and WO 2021/000684 disclose oxazolidinone antibiotics for the treatment of tuberculosis.

Development of efficient and low-cost processes to prepare oxazolidinone compounds would be desired in order to supply individuals in need of treatment of Mycobacteria tuberculosis worldwide.

### SUMMARY OF THE INVENTION

The invention is directed to novel crystalline forms of the methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, also represented by the compound of Formula I

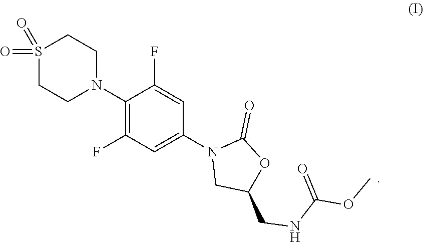 [SMILES: COC(=O)NC[C@H]1CN(c2cc(F)c(N3CCS(=O)(=O)CC3)c(F)c2)C(=O)O1]

Certain crystalline forms, have advantages in the preparation of pharmaceutical compositions of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, such as ease of processing, crystallization and handling. In particular, they exhibit improved physicochemical properties, such as stability to stress, rendering them particularly suitable for the manufacture of various pharmaceutical dosage forms. In addition, certain forms provide advantages in dosing.

The crystalline compounds described, and their pharmaceutically acceptable salts can be useful, for example, for the treatment or prevention of bacterial infections, for example, mycobacterial infections. The invention also concerns pharmaceutical compositions containing the novel crystalline forms of the compound of Formula I, as well as methods of using such forms for the treatment and prevention of mycobacterial infections such as those caused by Mycobacteria tuberculosis.

In certain embodiments, described herein are pharmaceutical compositions comprising crystalline methyl ({(5S)-3-[4-(1,1-dioxo-li-thiomorpholin-4-yl)-3,5-difluorophenyl]-2-oxo-1,3-oxazolidin-5-yl}methyl)carbamate and a pharmaceutically acceptable carrier.

The invention is directed to processes and intermediates for preparing crystalline forms of the compound of Formula I. More particularly, the invention includes processes for preparation of a compound of Formula I, or a pharmaceutically acceptable salt thereof:

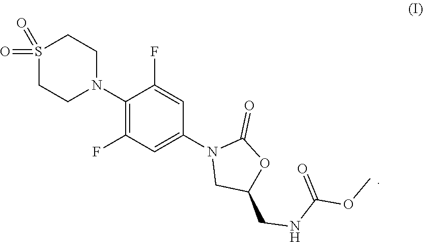 [SMILES: COC(=O)NC[C@H]1CN(c2cc(F)c(N3CCS(=O)(=O)CC3)c(F)c2)C(=O)O1]

Also described herein are pharmaceutical compositions comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate and a pharmaceutically acceptable carrier. An amorphous dispersion formulation of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can be made by hot melt extrusion (HME) with a polymer, which can be used in formulating pharmaceutical compositions comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

Embodiments, sub-embodiments and features of the invention are either further described herein or will be apparent from the ensuing description, examples and appended claims.

### BRIEF DESCRIPTION OF THE DRAWINGS

FIG. 1 is a graph of a Powder X-Ray Diffraction (PXRD) pattern of crystalline Form I of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the intensity of the peaks as defined by counts per second versus the diffraction angle 2 theta (2θ) in degrees.

FIG. 2 is a Thermogravimetric Analysis (TGA) curve of crystalline Form I of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the sample weight loss in percentage (%) as function of temperature in degree Celsius (° C.).

FIG. 3 is a Differential Scanning Calorimetry (DSC) curve of crystalline Form I of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the total heat flow (Q) in Watt per gram (W/g) as function of temperature in degree Celsius (° C.).

FIG. 4 is a graph of a Powder X-Ray Diffraction (PXRD) pattern of crystalline Form II of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the intensity of the peaks as defined by counts per second versus the diffraction angle 2 theta (2θ) in degrees.

FIG. 5 is a Thermogravimetric Analysis (TGA) curve of crystalline Form II of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the sample weight loss in percentage (%) as function of temperature in degree Celsius (° C.).

FIG. 6 is a Differential Scanning Calorimetry (DSC) curve of crystalline Form II of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the total heat flow (Q) in Watt per gram (W/g) as function of temperature in degree Celsius (° C.).

FIG. 7 is a graph of a Powder X-Ray Diffraction (PXRD) pattern of crystalline Type C of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the intensity of the peaks as defined by counts per second versus the diffraction angle 2 theta (2θ) in degrees.

FIG. 8 is a Thermogravimetric Analysis (TGA) curve of crystalline Type C of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the sample weight loss in percentage (%) as function of temperature in degree Celsius (° C.).

FIG. 9 is a Differential Scanning Calorimetry (DSC) curve of crystalline Type C of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the total heat flow (Q) in Watt per gram (W/g) as function of temperature in degree Celsius (° C.).

FIG. 10 is a graph of a Powder X-Ray Diffraction (PXRD) pattern of crystalline Form III of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the intensity of the peaks as defined by counts per second versus the diffraction angle 2 theta (2θ) in degrees.

FIG. 11 is a Thermogravimetric Analysis (TGA) curve of crystalline Form III of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the sample weight loss in percentage (%) as function of temperature in degree Celsius (° C.).

FIG. 12 is a Differential Scanning Calorimetry (DSC) curve of crystalline Form III of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, generated using the equipment and methods described herein. The graph plots the total heat flow (Q) in Watt per gram (W/g) as function of temperature in degree Celsius (° C.).

FIG. 13 is a graph overlaying the Powder X-Ray Diffraction (PXRD) patterns of experimental Form I and simulated Form I from SC-XRD. The graph plots the intensity of the peaks as defined by counts per second versus the diffraction angle 2 theta (2θ) in degrees.

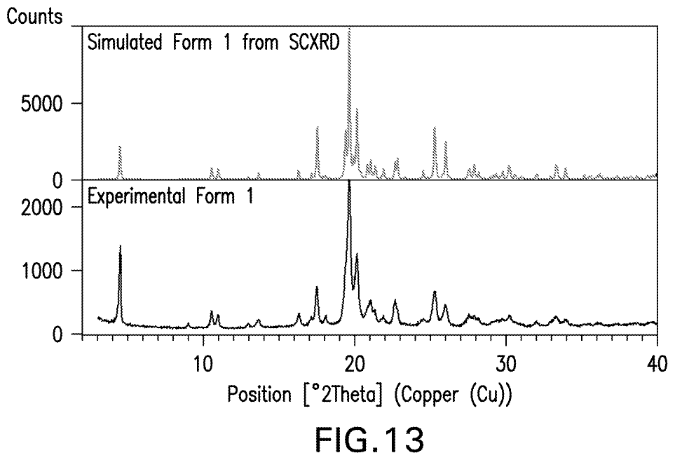

FIG. 14 is a graph overlaying the Powder X-Ray Diffraction (PXRD) patterns of experimental Form III and simulated Form III from SC-XRD. The graph plots the intensity of the peaks as defined by counts per second versus the diffraction angle 2 theta (2θ) in degrees.

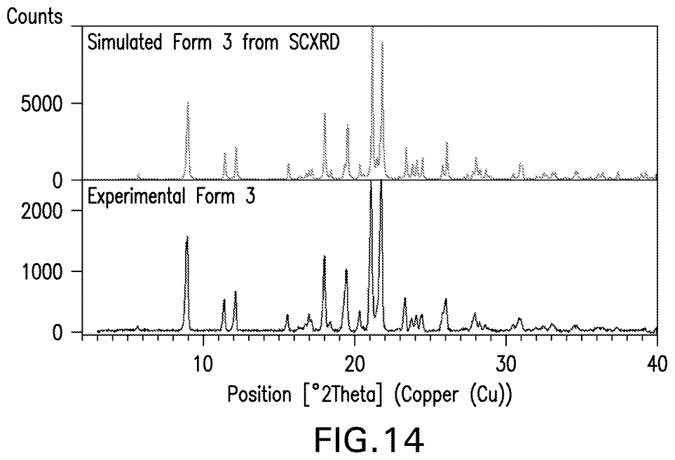

FIG. 15 is a graph showing results from a dog study comparing the PK of Form II and an amorphous formulation of the compound of Formula I. See Example 6.

### DETAILED DESCRIPTION OF THE INVENTION

### Definitions

The terms used herein have their ordinary meaning and the meaning of such terms is independent at each occurrence thereof. That notwithstanding and except where stated otherwise, the following definitions apply throughout the specification and claims.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. In some embodiments, pharmaceutically acceptable salts are ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, including a human, canine, feline, bovine, or porcine patient, preferably a human patient, receiving or about to receive medical treatment.

The term “treat” or “treatment” means to administer an agent, such as a composition containing any of the compounds described herein, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a mammalian subject with a disorder, disease or symptom, or with the potential to develop such a disorder, disease or symptom.

### Crystalline Forms of the Compound of Formula I

Described herein are crystalline forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, also represented as the compound of Formula I:

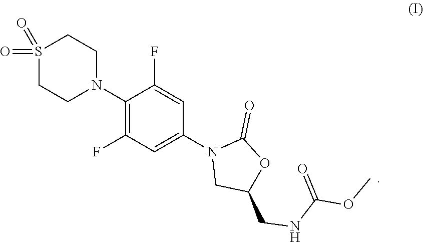 [SMILES: COC(=O)NC[C@H]1CN(c2cc(F)c(N3CCS(=O)(=O)CC3)c(F)c2)C(=O)O1]

Unless a specific form designation is given, the term “crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate” refers to all crystalline forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate described herein.

One embodiment of the crystalline forms described herein is methyl({(5S)-3-[4-(1,1-dioxo-1λ⁶-thiomorpholin-4-yl)-3,5-difluorophenyl]-2-oxo-1,3-oxazolidin-5-yl}methyl)carbamate (Form I). Form I is further described below.

Another embodiment of the crystalline forms described herein is methyl ({(5S)-3-[4-(1,1-dioxo-1λ⁶-thiomorpholin-4-yl)-3,5-difluorophenyl]-2-oxo-1,3-oxazolidin-5-yl}methyl)carbamate (Form II). Form II is further described below.

Another embodiment of the crystalline forms described herein is methyl ({(5S)-3-[4-(1,1-dioxo-1λ⁶-thiomorpholin-4-yl)-3,5-difluorophenyl]-2-oxo-1,3-oxazolidin-5-yl}methyl)carbamate (Type C). Type C is further described below.

Still another embodiment of the crystalline forms described herein is methyl ({(5S)-3-[4-(1,1-dioxo-1λ⁶-thiomorpholin-4-yl)-3,5-difluorophenyl]-2-oxo-1,3-oxazolidin-5-yl}methyl)carbamate (Form III). Form III is further described below.

A further embodiment of the invention provides a particular drug substance that comprises at least one of the crystalline forms described herein. By “drug substance” is meant the active pharmaceutical ingredient. The amount of crystalline form in the drug substance can be quantified by the use of physical methods such as X-ray powder diffraction, solid-state fluorine-19 magic-angle spinning (MAS) nuclear magnetic resonance spectroscopy, solid-state carbon-13 cross-polarization magic-angle spinning (CPMAS) nuclear magnetic resonance spectroscopy, solid state Fourier-transform infrared spectroscopy, and Raman spectroscopy.

In a class of this embodiment, a crystalline form of the compound of Formula I is present in about 5% to about 100% by weight of the drug substance. In a second class of this embodiment, a crystalline form of the compound of Formula I is present in about 10% to about 100% by weight of the drug substance. In a third class of this embodiment, a crystalline form of the compound of Formula I is present in about 25% to about 100% by weight of the drug substance. In a fourth class of this embodiment, a crystalline form of the compound of Formula I is present in about 50% to about 100% by weight of the drug substance. In a fifth class of this embodiment, a crystalline form of the compound of Formula I is present in about 75% to about 100% by weight of the drug substance. In a sixth class of this embodiment, substantially all of the drug substance is a crystalline form of the compound of Formula I, i.e., the drug substance is substantially phase pure crystalline.

In another class of this embodiment, at least 5% by weight of the drug substance is a crystalline form of the compound of Formula I. In a yet another class of this embodiment, at least 10% by weight of the drug substance is a crystalline form of the compound of Formula I. In a still another class of this embodiment, at least 15% by weight of the drug substance is a crystalline form of the compound of Formula I. In another class of this embodiment, at least 20% by weight of the drug substance is a crystalline form of the compound of Formula I. In yet another class of this embodiment, at least 25% by weight of the drug substance is a crystalline form of the compound of Formula I. In still another class of this embodiment, at least 30% by weight of the drug substance is a crystalline form of the compound of Formula I. In another class of this embodiment, at least 35% by weight of the drug substance is a crystalline form of the compound of Formula I. In a yet another class of this embodiment, at least 40% by weight of the drug substance is a crystalline form of the compound of Formula I. In a still another class of this embodiment, at least 45% by weight of the drug substance is a crystalline form of the compound of Formula I. In another class of this embodiment, at least 50% by weight of the drug substance is a crystalline form of the compound of Formula I. In yet another class of this embodiment, at least 55% by weight of the drug substance is a crystalline form of the compound of Formula I. In still another class of this embodiment, at least 60% by weight of the drug substance is a crystalline form of the compound of Formula I. In another class of this embodiment, at least 65% by weight of the drug substance is a crystalline form of the compound of Formula I. In a yet another class of this embodiment, at least 70% by weight of the drug substance is a crystalline form of the compound of Formula I. In a still another class of this embodiment, at least 75% by weight of the drug substance is a crystalline form of the compound of Formula I. In another class of this embodiment, at least 80% by weight of the drug substance is a crystalline form of the compound of Formula I. In yet another class of this embodiment, at least 85% by weight of the drug substance is a crystalline form of the compound of Formula I. In still another class of this embodiment, at least 90% by weight of the drug substance is a crystalline form of the compound of Formula I. In another class of this embodiment, at least 95% by weight of the drug substance is a crystalline form of the compound of Formula I. In a yet another class of this embodiment, at least 100% by weight of the drug substance is a crystalline form of the compound of Formula I.

In a class of this embodiment, crystalline Form I of the compound of Formula I is present in about 5% to about 100% by weight of the drug substance. In a second class of this embodiment, crystalline Form I of the compound of Formula I is present in about 10% to about 100% by weight of the drug substance. In a third class of this embodiment, crystalline Form I of the compound of Formula I is present in about 25% to about 100% by weight of the drug substance. In a fourth class of this embodiment, crystalline Form I of the compound of Formula I is present in about 50% to about 100% by weight of the drug substance. In a fifth class of this embodiment, crystalline Form I of the compound of Formula I is present in about 75% to about 100% by weight of the drug substance. In a sixth class of this embodiment, substantially all of the drug substance is crystalline Form I of the compound of Formula I, i.e., the drug substance is substantially phase pure crystalline.

In another class of this embodiment, at least 5% by weight of the drug substance is crystalline Form I of the compound of Formula I. In a yet another class of this embodiment, at least 10% by weight of the drug substance is crystalline Form I of the compound of Formula I. In a still another class of this embodiment, at least 15% by weight of the drug substance is crystalline Form I of the compound of Formula I. In another class of this embodiment, at least 20% by weight of the drug substance is crystalline Form I of the compound of Formula I. In yet another class of this embodiment, at least 25% by weight of the drug substance is crystalline Form I of the compound of Formula L In still another class of this embodiment, at least 30% by weight of the drug substance is crystalline Form I of the compound of Formula I. In another class of this embodiment, at least 35% by weight of the drug substance is crystalline Form I of the compound of Formula I. In a yet another class of this embodiment, at least 40% by weight of the drug substance is crystalline Form I of the compound of Formula I. In a still another class of this embodiment, at least 45% by weight of the drug substance is crystalline Form I of the compound of Formula I. In another class of this embodiment, at least 50% by weight of the drug substance is crystalline Form I of the compound of Formula I. In yet another class of this embodiment, at least 55% by weight of the drug substance is crystalline Form I of the compound of Formula I. In still another class of this embodiment, at least 60% by weight of the drug substance is crystalline Form I of the compound of Formula I. In another class of this embodiment, at least 65% by weight of the drug substance is crystalline Form I of the compound of Formula I. In a yet another class of this embodiment, at least 70% by weight of the drug substance is crystalline Form I of the compound of Formula I. In a still another class of this embodiment, at least 75% by weight of the drug substance is crystalline Form I of the compound of Formula I. In another class of this embodiment, at least 80% by weight of the drug substance is crystalline Form I of the compound of Formula I. In yet another class of this embodiment, at least 85% by weight of the drug substance is crystalline Form I of the compound of Formula I. In still another class of this embodiment, at least 90% by weight of the drug substance is crystalline Form I of the compound of Formula I. In another class of this embodiment, at least 95% by weight of the drug substance is crystalline Form I of the compound of Formula I. In a yet another class of this embodiment, about 100% by weight of the drug substance is crystalline Form I of the compound of Formula I. In another class of this embodiment, 100% by weight of the drug substance is crystalline Form I of the compound of Formula I.

In a class of this embodiment, crystalline Form II of the compound of Formula I is present in about 5% to about 100% by weight of the drug substance. In a second class of this embodiment, crystalline Form II of the compound of Formula I is present in about 10% to about 100% by weight of the drug substance. In a third class of this embodiment, crystalline Form II of the compound of Formula I is present in about 25% to about 100% by weight of the drug substance. In a fourth class of this embodiment, crystalline Form II of the compound of Formula I is present in about 50% to about 100% by weight of the drug substance. In a fifth class of this embodiment, crystalline Form II of the compound of Formula I is present in about 75% to about 100% by weight of the drug substance. In a sixth class of this embodiment, substantially all of the drug substance is crystalline Form II of the compound of Formula I, i.e., the drug substance is substantially phase pure crystalline.

In another class of this embodiment, at least 5% by weight of the drug substance is crystalline Form II of the compound of Formula I. In a yet another class of this embodiment, at least 10% by weight of the drug substance is crystalline Form II of the compound of Formula I. In a still another class of this embodiment, at least 15% by weight of the drug substance is crystalline Form II of the compound of Formula I. In another class of this embodiment, at least 20% by weight of the drug substance is crystalline Form II of the compound of Formula I. In yet another class of this embodiment, at least 25% by weight of the drug substance is crystalline Form II of the compound of Formula I. In still another class of this embodiment, at least 30% by weight of the drug substance is crystalline Form II of the compound of Formula I. In another class of this embodiment, at least 35% by weight of the drug substance is crystalline Form II of the compound of Formula I. In a yet another class of this embodiment, at least 40% by weight of the drug substance is crystalline Form II of the compound of Formula I. In a still another class of this embodiment, at least 45% by weight of the drug substance is crystalline Form II of the compound of Formula I. In another class of this embodiment, at least 50% by weight of the drug substance is crystalline Form II of the compound of Formula I. In yet another class of this embodiment, at least 55% by weight of the drug substance is crystalline Form II of the compound of Formula I. In still another class of this embodiment, at least 60% by weight of the drug substance is crystalline Form II of the compound of Formula I. In another class of this embodiment, at least 65% by weight of the drug substance is crystalline Form II of the compound of Formula I. In a yet another class of this embodiment, at least 70% by weight of the drug substance is crystalline Form II of the compound of Formula I. In a still another class of this embodiment, at least 75% by weight of the drug substance is crystalline Form II of the compound of Formula I. In another class of this embodiment, at least 80% by weight of the drug substance is crystalline Form II of the compound of Formula I. In yet another class of this embodiment, at least 85% by weight of the drug substance is crystalline Form II of the compound of Formula I. In still another class of this embodiment, at least 90% by weight of the drug substance is crystalline Form II of the compound of Formula I. In another class of this embodiment, at least 95% by weight of the drug substance is crystalline Form II of the compound of Formula I. In a yet another class of this embodiment, about 100% by weight of the drug substance is crystalline Form II of the compound of Formula I. In another class of this embodiment, 100% by weight of the drug substance is crystalline Form II of the compound of Formula I.

In a class of this embodiment, crystalline Type C of the compound of Formula I is present in about 5% to about 100% by weight of the drug substance. In a second class of this embodiment, crystalline Type C of the compound of Formula I is present in about 10% to about 100% by weight of the drug substance. In a third class of this embodiment, crystalline Type C of the compound of Formula I is present in about 25% to about 100% by weight of the drug substance. In a fourth class of this embodiment, crystalline Type C of the compound of Formula I is present in about 50% to about 100% by weight of the drug substance. In a fifth class of this embodiment, crystalline Type C of the compound of Formula I is present in about 75% to about 100% by weight of the drug substance. In a sixth class of this embodiment, substantially all of the drug substance is crystalline Type C of the compound of Formula I. i.e., the drug substance is substantially phase pure crystalline.

In another class of this embodiment, at least 5% by weight of the drug substance is crystalline Type C of the compound of Formula I. In a yet another class of this embodiment, at least 10% by weight of the drug substance is crystalline Type C of the compound of Formula I. In a still another class of this embodiment, at least 15% by weight of the drug substance is crystalline Type C of the compound of Formula I. In another class of this embodiment, at least 20% by weight of the drug substance is crystalline Type C of the compound of Formula I. In yet another class of this embodiment, at least 25% by weight of the drug substance is crystalline Type C of the compound of Formula I. In still another class of this embodiment, at least 30% by weight of the drug substance is crystalline Type C of the compound of Formula I. In another class of this embodiment, at least 35% by weight of the drug substance is crystalline Type C of the compound of Formula I. In a yet another class of this embodiment, at least 40% by weight of the drug substance is crystalline Type C of the compound of Formula I. In a still another class of this embodiment, at least 45% by weight of the drug substance is crystalline Type C of the compound of Formula I. In another class of this embodiment, at least 50% by weight of the drug substance is crystalline Type C of the compound of Formula I. In yet another class of this embodiment, at least 55% by weight of the drug substance is crystalline Type C of the compound of Formula I. In still another class of this embodiment, at least 60% by weight of the drug substance is crystalline Type C of the compound of Formula I. In another class of this embodiment, at least 65% by weight of the drug substance is crystalline Type C of the compound of Formula I. In a yet another class of this embodiment, at least 70% by weight of the drug substance is crystalline Type C of the compound of Formula I. In a still another class of this embodiment, at least 75% by weight of the drug substance is crystalline Type C of the compound of Formula I. In another class of this embodiment, at least 80% by weight of the drug substance is crystalline Type C of the compound of Formula I. In yet another class of this embodiment, at least 85% by weight of the drug substance is crystalline Type C of the compound of Formula I. In still another class of this embodiment, at least 90% by weight of the drug substance is crystalline Type C of the compound of Formula I. In another class of this embodiment, at least 95% by weight of the drug substance is crystalline Type C of the compound of Formula I. In a yet another class of this embodiment, about 100% by weight of the drug substance is crystalline Type C of the compound of Formula I. In another class of this embodiment, 100% by weight of the drug substance is crystalline Type C of the compound of Formula I.

In a class of this embodiment, crystalline Form III of the compound of Formula I is present in about 5% to about 100% by weight of the drug substance. In a second class of this embodiment, crystalline Form III of the compound of Formula I is present in about 10% to about 100% by weight of the drug substance. In a third class of this embodiment, crystalline Form III of the compound of Formula I is present in about 25% to about 100% by weight of the drug substance. In a fourth class of this embodiment, crystalline Form III of the compound of Formula I is present in about 50% to about 100% by weight of the drug substance. In a fifth class of this embodiment, crystalline Form III of the compound of Formula I is present in about 75% to about 100% by weight of the drug substance. In a sixth class of this embodiment, substantially all of the drug substance is crystalline Form III of the compound of Formula I, i.e., the drug substance is substantially phase pure crystalline.

In another class of this embodiment, at least 5% by weight of the drug substance is crystalline Form III of the compound of Formula I. In a yet another class of this embodiment, at least 10% by weight of the drug substance is crystalline Form III of the compound of Formula I. In a still another class of this embodiment, at least 15% by weight of the drug substance is crystalline Form III of the compound of Formula I. In another class of this embodiment, at least 20% by weight of the drug substance is crystalline Form III of the compound of Formula I. In yet another class of this embodiment, at least 25% by weight of the drug substance is crystalline Form III of the compound of Formula I. In still another class of this embodiment, at least 30% by weight of the drug substance is crystalline Form III of the compound of Formula I. In another class of this embodiment, at least 35% by weight of the drug substance is crystalline Form III of the compound of Formula I. In a yet another class of this embodiment, at least 40% by weight of the drug substance is crystalline Form III of the compound of Formula I. In a still another class of this embodiment, at least 45% by weight of the drug substance is crystalline Form III of the compound of Formula I. In another class of this embodiment, at least 50% by weight of the drug substance is crystalline Form III of the compound of Formula I. In yet another class of this embodiment, at least 55% by weight of the drug substance is crystalline Form III of the compound of Formula I. In still another class of this embodiment, at least 60% by weight of the drug substance is crystalline Form III of the compound of Formula I. In another class of this embodiment, at least 65% by weight of the drug substance is crystalline Form III of the compound of Formula I. In a yet another class of this embodiment, at least 70% by weight of the drug substance is crystalline Form III of the compound of Formula I. In a still another class of this embodiment, at least 75% by weight of the drug substance is crystalline Form III of the compound of Formula I. In another class of this embodiment, at least 80% by weight of the drug substance is crystalline Form III of the compound of Formula I. In yet another class of this embodiment, at least 85% by weight of the drug substance is crystalline Form III of the compound of Formula I. In still another class of this embodiment, at least 90% by weight of the drug substance is crystalline Form III of the compound of Formula I. In another class of this embodiment, at least 95% by weight of the drug substance is crystalline Form III of the compound of Formula I. In a yet another class of this embodiment, about 100% by weight of the drug substance is crystalline Form III of the compound of Formula I. In another class of this embodiment, 100% by weight of the drug substance is crystalline Form III of the compound of Formula I.

The crystalline compounds described, and their pharmaceutically acceptable salts can be useful, for example, for the treatment or prevention of bacterial infections, for example, mycobacterial infections. The crystalline compounds described, and their pharmaceutically acceptable salts can be useful, for example, for the treatment or prevention of bacterial infections, for example, mycobacterial infections, such as those caused by Mycobacteria tuberculosis.

Another aspect of the invention provides a method for the prevention or treatment of bacterial infections, for example, mycobacterial infections, such as those caused by Mycobacteria tuberculosis, which method comprises administering to a patient in need of such prevention or treatment a prophylactically or therapeutically effective amount of a crystalline form of the compound of Formula I.

Another aspect of the invention provides a method for the treatment of bacterial infections, for example, mycobacterial infections, such as those caused by Mycobacteria tuberculosis, which method comprises administering to a patient in need of such prevention or treatment a prophylactically or therapeutically effective amount of a crystalline form of the compound of Formula I.

Another aspect of the invention provides a method for the prevention of bacterial infections, for example, mycobacterial infections, such as those caused by Mycobacteria tuberculosis, which method comprises administering to a patient in need of such prevention or treatment a prophylactically or therapeutically effective amount of a crystalline form of the compound of Formula I.

The invention also provides the use of a crystalline form of the compound of Formula I for the prevention or treatment in a patient of bacterial infections, for example, mycobacterial infections, such as those caused by Mycobacteria tuberculosis,

The invention also provides the use of a crystalline form of the compound of Formula I for the manufacture of a medicament for the prevention or treatment in a patient of bacterial infections, for example, mycobacterial infections, such as those caused by Mycobacteria tuberculosis.

The invention also provides the use of a crystalline form of the compound of Formula I for the treatment in a patient of bacterial infections, for example, mycobacterial infections, such as those caused by Mycobacteria tuberculosis,

The invention also provides the use of a crystalline form of the compound of Formula I for the manufacture of a medicament for the treatment in a patient of bacterial infections, for example, mycobacterial infections, such as those caused by Mycobacteria tuberculosis.

The invention also provides the use of a crystalline form of the compound of Formula I for the prevention in a patient of bacterial infections, for example, mycobacterial infections, such as those caused by Mycobacteria tuberculosis,

The invention also provides the use of a crystalline form of the compound of Formula I for the manufacture of a medicament for the prevention in a patient of bacterial infections, for example, mycobacterial infections, such as those caused by Mycobacteria tuberculosis.

The invention also provides pharmaceutical compositions comprising a crystalline form described herein, in association with one or more pharmaceutically acceptable carriers or excipients. The invention also provides pharmaceutical compositions comprising crystalline Form I described herein, in association with one or more pharmaceutically acceptable carriers or excipients. The invention also provides pharmaceutical compositions comprising crystalline Form II described herein, in association with one or more pharmaceutically acceptable carriers or excipients. The invention also provides pharmaceutical compositions comprising crystalline Type C described herein, in association with one or more pharmaceutically acceptable carriers or excipients. The invention also provides pharmaceutical compositions comprising crystalline Form III described herein, in association with one or more pharmaceutically acceptable carriers or excipients. In one embodiment the pharmaceutical composition comprises a therapeutically effective amount of the active pharmaceutical ingredient in admixture with one or more pharmaceutically acceptable excipients wherein the active pharmaceutical ingredient comprises a detectable amount of a crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

In a second embodiment, the pharmaceutical composition comprises a therapeutically effective amount of the active pharmaceutical ingredient in an admixture with one or more pharmaceutically acceptable excipients wherein the active pharmaceutical ingredient comprises about 1% to about 100% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a class of this second embodiment, the active pharmaceutical ingredient in such compositions comprises about 5% to about 100% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a second class of this embodiment, the active pharmaceutical ingredient in such compositions comprises about 10% to about 100% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a third class of this embodiment, the active pharmaceutical ingredient in such compositions comprises about 25% to about 100% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a fourth class of this embodiment, the active pharmaceutical ingredient in such compositions comprises about 50% to about 100% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

In a third embodiment the pharmaceutical composition comprises a therapeutically effective amount of the active pharmaceutical ingredient in an admixture one or more with pharmaceutically acceptable excipients wherein the active pharmaceutical ingredient comprises at least 1% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a class of this second embodiment, the active pharmaceutical ingredient in such compositions comprises at least 5% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a second class of this embodiment, the active pharmaceutical ingredient in such compositions comprises at least 10% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a third class of this embodiment, the active pharmaceutical ingredient in such compositions comprises at least 25% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a fourth class of this embodiment, the active pharmaceutical ingredient in such compositions comprises at least 50% by weight of crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be affected by methods known from the art, for example, as described in Remington's Pharmaceutical Sciences, 17th ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the invention, when used for the indicated effects, will range between about 0.01 mg per kg of body weight per day (mg/kg/day) to about 100 mg/kg/day, in some embodiments some, some doses will range from between 0.01 to 10 mg/kg/day, and in other embodiments some, some doses will range from between 0.1 to 5.0 mg/kg/day. For oral administration, the compositions are provided, in some embodiments, in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, and in some embodiments, from about 1 mg to about 200 mg of active ingredient. Intravenously, in some embodiments, doses will range from about 0.1 to about 10 mg/kg/minute during a constant rate infusion. The crystalline forms of the invention may be administered in a single daily dose, or the total daily dosage may be administered in divided doses of two, three or four times daily. However, methyl ({(5S)-3-[4-(1,1-dioxo-1λ⁶-thiomorpholin-4-yl)-3,5-difluorophenyl]-2-oxo-1,3-oxazolidin-5-yl}methyl)carbamate is a long acting anti-bacterial agent. Advantageously, the crystalline forms of the invention may be administered in a single weekly dose.

Furthermore, the crystalline forms of the invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the invention, the crystalline forms described herein (e.g., Form I, Form II, Form III, and Type C) can form the active pharmaceutical ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

### Processes and Intermediates for Preparing Crystalline Forms Compounds of Formula I

The invention also relates to processes for preparing a compound of Formula I

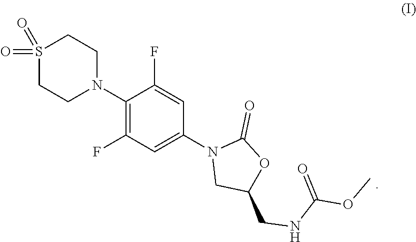 [SMILES: COC(=O)NC[C@H]1CN(c2cc(F)c(N3CCS(=O)(=O)CC3)c(F)c2)C(=O)O1]

In certain embodiments, invention relates to processes for preparing crystalline forms of the compound of Formula I

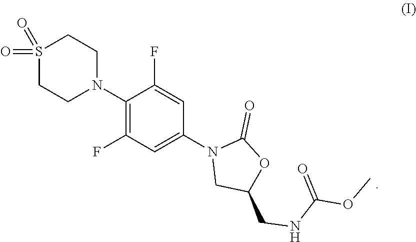 [SMILES: COC(=O)NC[C@H]1CN(c2cc(F)c(N3CCS(=O)(=O)CC3)c(F)c2)C(=O)O1]

The invention also relates to processes for preparing a compound of Formula I

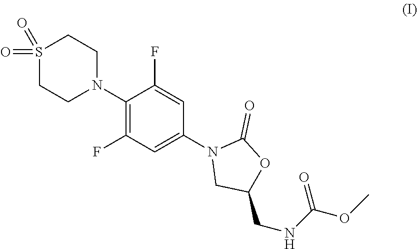 [SMILES: COC(=O)NC[C@H]1CN(c2cc(F)c(N3CCS(=O)(=O)CC3)c(F)c2)C(=O)O1]

via a convergent synthesis. In certain embodiments, the processes described herein, eliminate the protecting group swap on the oxazolidinone amine side chain, which is required when typical reagents such as (S)-1-acetamido-3-chloropropan-2-yl acetate is used to form the oxazolidinone ring, as described in WO 2021/000684.

The following process is disclosed in WO 2021/000684

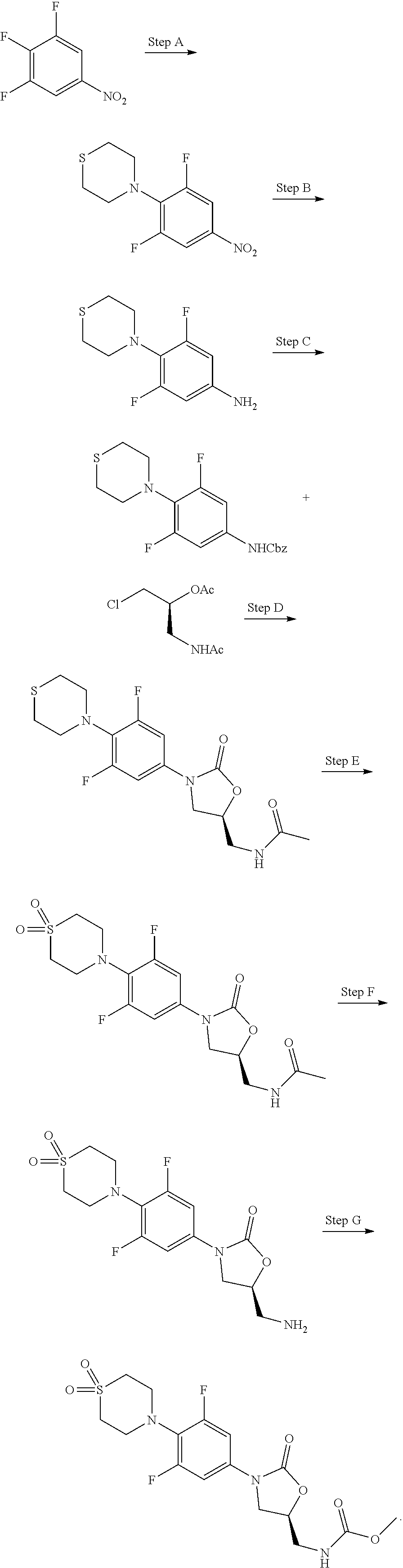

In Step D, methanol and lithium tert-butoxide were added to a solution of benzyl [3,5-difluoro-4-(thiomorpholin-4-yl)phenyl]carbamate in tetrahydrofuran at 0° C., and the reaction mixture allowed to stir for 1 hour. (2S)-1-acetamido-3-chloropropan-2-yl acetate was added and the reaction mixture allowed to warm to ambient temperature and was stirred for 16 hours. The reaction mixture was adjusted to approximately pH 6 by addition of an aqueous solution of HCl (1 M) and concentrated under reduced pressure. The residue was extracted with dichloromethane (3×30 mL), and the combined organic layers were dried over sodium sulfate, filtered, and the filtrate concentrated under reduced pressure. The residue was purified by silica gel column chromatography, eluting with a gradient of ethyl acetate:petroleum ether.

In Step E, a solution of Oxone in water was added to a solution of N-({(5S)-3-[3,5-difluoro-4-(thiomorpholin-4-yl)phenyl]-2-oxo-1,3-oxazolidin-5-yl}methyl)acetamide in methanol at 0° C., and the reaction mixture was allowed to warm to ambient temperature and was stirred for 4 hours. A saturated aqueous solution of sodium sulfite was added and the mixture was concentrated under reduced pressure. The residue was extracted with dichloromethane and the combined organic layers were dried over sodium sulfate, filtered, and the filtrate was concentrated under reduced pressure to afford the product in sufficient purity for use in Step F.

In Step F, water and a concentrated aqueous solution of HCl were added to a solution of N-({(5S)-3-[4-(1,1-dioxo-1λ⁶-thiomorpholin-4-yl)-3,5-difluorophenyl]-2-oxo-1,3-oxazolidin-5-yl}methyl)acetamide in methanol. The reaction mixture was warmed to 70° C. and allowed to stir for 1.5 days. The reaction mixture was cooled and concentrated under reduced pressure to afford the title compound in sufficient purity for use in Step G.

In Step G, N,N-diisopropylethylamine and methyl chloroformate were added to a solution of 4-{4-[(5S)-5-(aminomethyl)-2-oxo-1,3-oxazolidin-3-yl]-2,6-difluorophenyl}-1λ⁶-thiomorpholine-1,1-dione in dichloromethane at 0° C. The reaction mixture was allowed to warm to ambient temperature and allowed to stir for 1 hour. The reaction mixture was concentrated under reduced pressure, and the residue purified by preparative HPLC, eluting with a gradient of acetonitrile:water containing 0.1% trifluoroacetic acid to provide the compound of Formula I.

The methods of preparing the compound of Formula I described herein, eliminate the protecting group swap on the oxazolidinone amine side chain as shown described in Steps F and G.

In the methods of preparing a compound of Formula I described herein, methyl (S)-(3-chloro-2-hydroxypropyl)carbamate (Formula 6) is reacted with compounds of Formula 5a or Formula 5b under the disclosed conditions, giving a much cleaner reaction compared to the reaction when the bis-protected reagent (S)-1-chloro-3-((methoxycarbonyl)amino)propan-2-yl acetate is used.

In certain embodiments, in the methods of preparing a compound of Formula I described herein, the oxazolidinone ring with a methyl carbamate side chain was formed directly from the reaction between benzyl (4-(1,1-dioxidothiomorpholino)-3,5-difluorophenyl)carbamate (Formula 5a) or methyl (4-(1,1-dioxidothiomorpholino)-3,5-difluorophenyl)carbamate (Formula 5b).

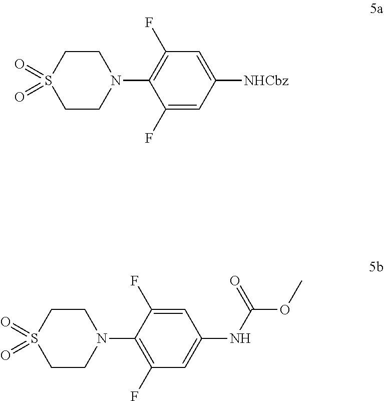 [SMILES: O=C(Nc1cc(F)c(N2CCS(=O)(=O)CC2)c(F)c1)OCc1ccccc1 ; COC(=O)Nc1cc(F)c(N2CCS(=O)(=O)CC2)c(F)c1]

and methyl (S)-(3-chloro-2-hydroxypropyl)carbamate (Formula 6)

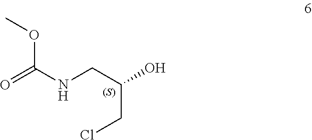

to give the compound of Formula I.

In certain embodiments, the process is performed in the presence of base. Suitable bases include, but are not limited to, sodium methoxide, potassium methoxide, lithium methoxide sodium t-butoxide, potassium t-butoxide and lithium t-butoxide. In certain embodiments, the base is sodium t-butoxide. In certain embodiments, the base is potassium t-butoxide. In certain embodiments, the base is lithium t-butoxide.

Also described herein are methods of preparing compounds of Formula 5a and 5b In certain embodiments, compounds of Formula 5a and 5b can be prepared via 3 steps: an SNAr reaction between 1,2,3-trifluoro-5-nitrobenzene (1) and thiomorpholine 1,1-dioxide hydrochloride (2) under basic conditions, a reduction of 4-(2,6-difluoro-4-nitrophenyl)thiomorpholine 1,1-dioxide (3) to form 4-(4-amino-2,6-difluorophenyl)thiomorpholine 1,1-dioxide (4), followed by a carbamate formation.

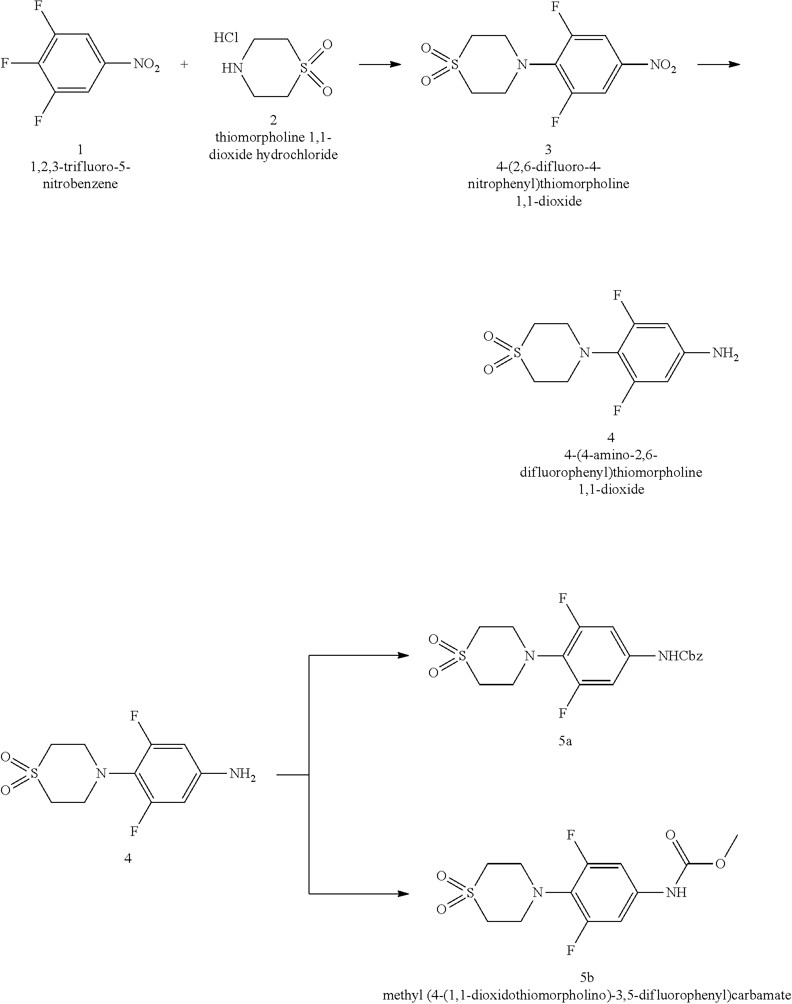

Also described herein are compounds of Formula 6

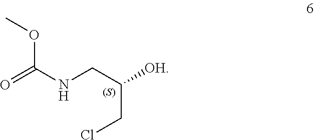

Also described herein are processes of preparing compounds of Formula 6. Methyl (S)-(3-chloro-2-hydroxypropyl)carbamate (Formula 6) can be accessed from (S)-1-amino-3-chloropropan-2-ol hydrochloride, either with methyl carbonochloridate or with dimethyl carbonate.

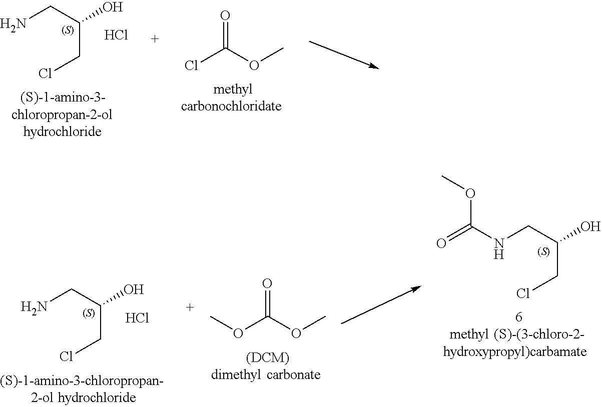

Also described herein are processes of making crystalline Form I or Form II of the compound of Formula I, or a pharmaceutically acceptable salt thereof, comprising treating a compound of Formula 5a or 5b with methyl (S)-(3-chloro-2-hydroxypropyl)carbamate (Formula 6).

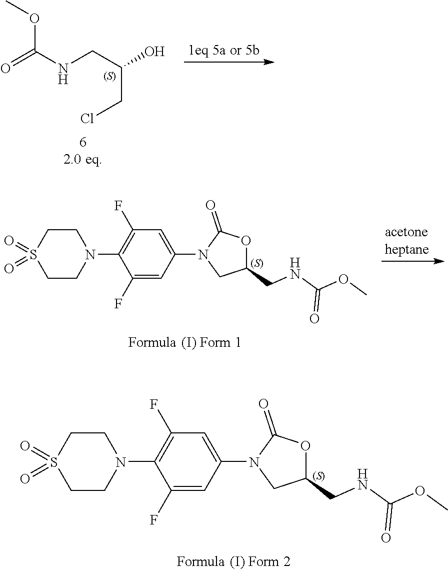

### Pharmaceutical Compositions of Amorphous Form of the Compound of Formula I

As described above and in the Examples below, methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can exist in anhydrous crystalline forms designated as Form I, Form II and Form III and Type C. An amorphous dispersion formulation of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can also be made by hot melt extrusion (HME) with a polymer, such as hydroxypropyl methyl cellulose acetate succinate (also known as HPMCAS, “Hypromellose acetate succinate), hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone or polyvinylpyrrolidinone-polyvinylacetate copolymers.

Also described herein are amorphous dispersion formulations comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In certain embodiments, amorphous dispersion formulations comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate are made by using hot melt extrusion of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate with hydroxypropyl methyl cellulose acetate succinate (HPMCAS-M).

In another embodiment, the amorphous dispersion formulation is made by hot melt extrusion with of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate polyvinylpyrrolidinone (PVP VA64®). One advantage of amorphous formulation of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate, is that the bioavailability of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate is improved.

The formulations comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate described, can be useful, for example, for the treatment or prevention of bacterial infections, for example, mycobacterial infections. The amorphous formulations described can be useful, for example, for the treatment or prevention of bacterial infections, for example, mycobacterial infections, such as those caused by Mycobacteria tuberculosis.

Another aspect of the invention provides a method for the prevention or treatment of bacterial infections, for example, mycobacterial infections, such as those caused by Mycobacteria tuberculosis, which method comprises administering to a patient in need of such prevention or treatment a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

Another aspect of the invention provides a method for the treatment of bacterial infections, for example, mycobacterial infections, such as those caused by Mycobacteria tuberculosis, which method comprises administering to a patient in need of such prevention or treatment a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

Another aspect of the invention provides a method for the prevention of bacterial infections, for example, mycobacterial infections, such as those caused by Mycobacteria tuberculosis, which method comprises administering to a patient in need of such prevention or treatment pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the prevention or treatment in a patient of bacterial infections, for example, mycobacterial infections, such as those caused by Mycobacteria tuberculosis.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the manufacture of a medicament for the prevention or treatment in a patient of bacterial infections, for example, mycobacterial infections, such as those caused by Mycobacteria tuberculosis.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the treatment in a patient of bacterial infections, for example, mycobacterial infections, such as those caused by Mycobacteria tuberculosis.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the manufacture of a medicament for the treatment in a patient of bacterial infections, for example, mycobacterial infections, such as those caused by Mycobacteria tuberculosis.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the prevention in a patient of bacterial infections, for example, mycobacterial infections, such as those caused by Mycobacteria tuberculosis.

The invention also provides for the use of a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate for the manufacture of a medicament for the prevention in a patient of bacterial infections, for example, mycobacterial infections, such as those caused by Mycobacteria tuberculosis.

The invention also provides pharmaceutical compositions comprising a pharmaceutical formulation comprising amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate described herein, in association with one or more pharmaceutically acceptable carriers or excipients.

In a second embodiment the pharmaceutical composition comprises a therapeutically effective amount of the active pharmaceutical ingredient in an admixture with one or more pharmaceutically acceptable excipients wherein the active pharmaceutical ingredient comprises about 1% to about 100% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a class of this second embodiment, the active pharmaceutical ingredient in such compositions comprises about 5% to about 100% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a second class of this embodiment, the active pharmaceutical ingredient in such compositions comprises about 10% to about 100% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a third class of this embodiment, the active pharmaceutical ingredient in such compositions comprises about 25% to about 100% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a fourth class of this embodiment, the active pharmaceutical ingredient in such compositions comprises about 50% to about 100% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

In a third embodiment the pharmaceutical composition comprises a therapeutically effective amount of the active pharmaceutical ingredient in an admixture with one or more pharmaceutically acceptable excipients wherein the active pharmaceutical ingredient comprises at least 1% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a class of this second embodiment, the active pharmaceutical ingredient in such compositions comprises about 5% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a second class of this embodiment, the active pharmaceutical ingredient in such compositions comprises at least 10% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a third class of this embodiment, the active pharmaceutical ingredient in such compositions comprises at least 25% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate. In a fourth class of this embodiment, the active pharmaceutical ingredient in such compositions comprises at least 50% by weight of amorphous methyl ({(5S)-3-[4-(1,1-dioxo-1λ⁶-thiomorpholin-4-yl)-3,5-difluorophenyl]-2-oxo-1,3-oxazolidin-5-yl}methyl)carbamate.

The compositions in accordance with the invention are suitably in unit dosage forms such as tablets, pills, capsules, powders, granules, sterile solutions or suspensions, metered aerosol or liquid sprays, drops, ampoules, auto-injector devices or suppositories. The compositions are intended for oral, parenteral, intranasal, sublingual, or rectal administration, or for administration by inhalation or insufflation. Formulation of the compositions according to the invention can conveniently be affected by methods known from the art, for example, as described in Remington's Pharmaceutical Sciences, 17th ed., 1995.

The dosage regimen is selected in accordance with a variety of factors including type, species, age, weight, sex and medical condition of the patient; the severity of the condition to be treated; the route of administration; and the renal and hepatic function of the patient. An ordinarily skilled physician, veterinarian, or clinician can readily determine and prescribe the effective amount of the drug required to prevent, counter or arrest the progress of the condition.

Oral dosages of the compounds of the invention, when used for the indicated effects, will range between about 0.01 mg per kg of body weight per day (mg/kg/day) to about 100 mg/kg/day, in some embodiments 0.01 to 10 mg/kg/day, and in other embodiments 0.1 to 5.0 mg/kg/day. For oral administration, the compositions are provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, and in some embodiments, from about 1 mg to about 200 mg of active ingredient. Intravenously, in some embodiments, doses will range from about 0.1 to about 10 mg/kg/minute during a constant rate infusion. The crystalline forms of the invention may be administered in a single daily dose, or the total daily dosage may be administered in divided doses of two, three or four times daily. However, methyl ({(5S)-3-[4-(1,1-dioxo-1λ⁶-thiomorpholin-4-yl)-3,5-difluorophenyl]-2-oxo-1,3-oxazolidin-5-yl}methyl)carbamate is a long acting anti-bacterial agent. Advantageously, the amorphous forms of the compound of the invention may be administered in a single weekly dose.

Furthermore, the amorphous form of the compound of the invention (i.e., amorphous form of the compound of Formula I) can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the invention, the amorphous form of the compound of Formula I described herein can form the active pharmaceutical ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug component can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

### EXAMPLES

The meanings of the abbreviations in Examples are shown below.
Acetonitrile=MeCN
Benzyl chloroformate=Cbz-Cl
Carboxybenzyl=Cbz
Dichloromethane=DCM
Diethylamine=DEA
Dimethyl carbamate=DMC
4-Dimethylaminopyridine=DMAP
Dimethyl sulfoxide=DMSO
Hydrochloric acid=HCl
Lithium tert-butoxide=t-BuOLi
N,N-diisopropylethylamine=DIPEA
N,N-dimethylformate=DMF
Tetrahydrofuran=THF
° C. means degrees Celsius
FIG (or FIG. or Fig. or Fig or fig. or fig) means Figure (or figure) and refers to the corresponding figure
g means gram (or grams)
mg means milligram (or milligrams)
% means percentage
mL means milliliter (or milliliters)
Q means heat
H means enthalpy
W means watt
J means joule
V means volume
“PXRD” is an abbreviation for powder x-ray diffraction
“SCXRD” is an abbreviation for single crystal x-ray diffraction
“TGA” is an abbreviation for thermogravimetric analysis
“DSC” is an abbreviation for differential scanning calorimetry

### Example 1

### Step 1

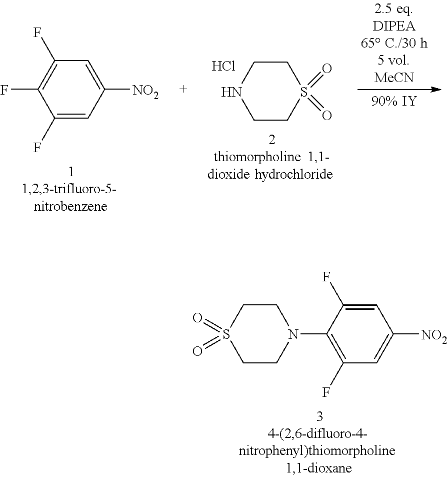

### 4-(2,6-difluoro-4-nitrophenyl)thiomorpholine 1,1-dioxide (3)

In a three-necked round bottom flask, a mixture of 1,2,3-trifluoro-5-nitrobenzene (1), thiomorpholine 1,1-dioxide hydrochloride (2), N,N-diisopropylethylamine (DIPEA) and acetonitrile (MeCN) was heated to 65° C. and stirred for 30 h. The resulting suspension was cooled to 25° C. and filtered. The wet cake was washed with acetonitrile and then dried to give 4-(2,6-difluoro-4-nitrophenyl)thiomorpholine 1,1-dioxide (3).

### Step 2

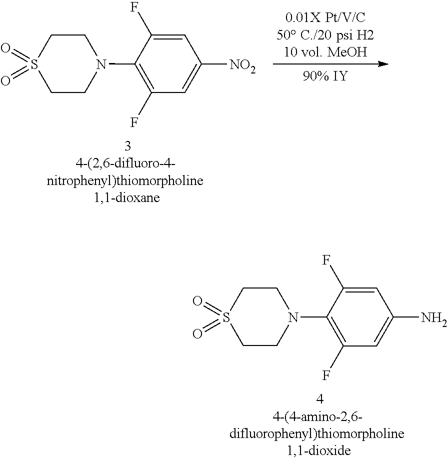

### 4-(4-amino-2,6-difluorophenyl)thiomorpholine 1,1-dioxide (4)

1% Platinum-vanadium on carbon was added to a stirred solution of 4-(2,6-difluoro-4-nitrophenyl)thiomorpholine 1,1-dioxide (3) in methanol at 25° C. The reaction mixture was placed under an atmosphere of hydrogen at (20 psi). The batch was heated to 50° C. under the atmosphere of hydrogen and stirred for 16 h. The reaction mixture was degassed and filtered through celite. Methanol was used to rinse the celite which was combined with filtrate. The solution was concentrated under reduced pressure and the crude was recrystallized from acetonitrile/water to give 4-(4-amino-2,6-difluorophenyl)thiomorpholine 1,1-dioxide (4).

### Step 3

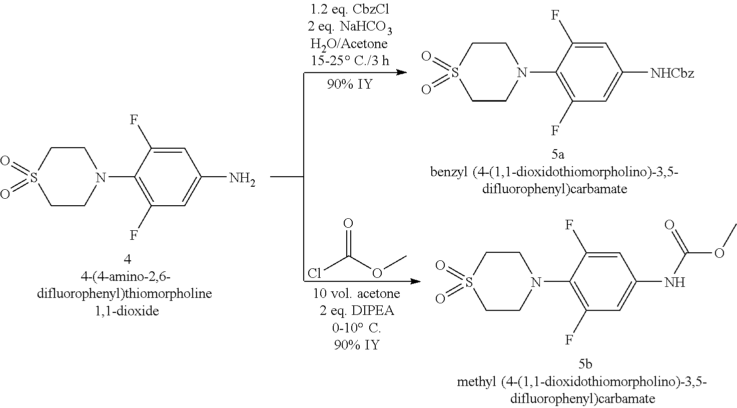

### benzyl (4-(1,1-dioxidothiomorpholino)-3,5-difluorophenyl)carbamate (5a)

Benzyl chloroformate (Cbz-Cl) was added dropwise to a solution of 4-(4-amino-2,6-difluorophenyl)thiomorpholine 1,1-dioxide (4, 100 g, 1.0 equiv.) in 5% aqueous bicarbonate (1264 g) and acetone (1000 mL) that was cooled to below 10° C. The batch was then heated to 20° C. and stirred for 3 h. The slurry was filtered and the crude was recrystallized from acetone/water to give benzyl (4-(1,1-dioxidothiomorpholino)-3,5-difluorophenyl)carbamate (5a).

### methyl (4-(1,1-dioxidothiomorpholino)-3,5-difluorophenyl)carbamate (5b)

Methyl chloroformate (117 g, 1.8 equiv.) was added dropwise to a jacket reactor containing a solution of 4-(4-amino-2,6-difluorophenyl)thiomorpholine 1,1-dioxide (4, 180 g, 1.0 equiv.) and N,N-diisopropylethylamine (177 g, 2.0 equiv.) in acetone (2100 mL) at 5° C. The reaction mixture was stirred for 1 h at 5° C. and quenched by water (2100 mL). The suspension was filtered and washed with washed with acetone/water (360 mL/360 mL). The wet cake was dried via vacuum to afford methyl (4-(1,1-dioxidothiomorpholino)-3,5-difluorophenyl)carbamate (5b).

### Step 4

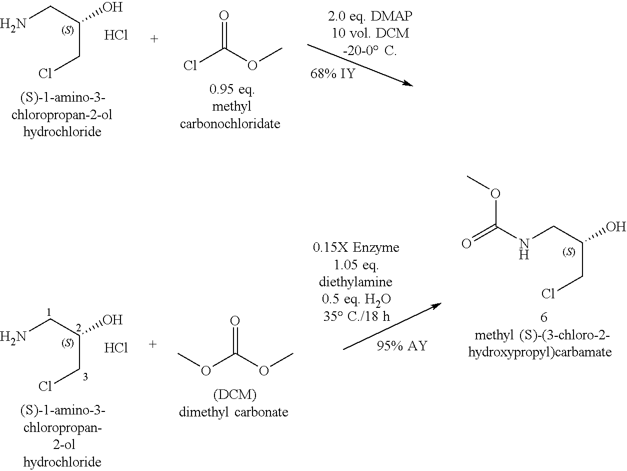

DMAP was added into a suspension of (S)-1-amino-3-chloropropan-2-ol hydrochloride in DCM, followed by methyl chloroformate (30.7 g, 0.95 equiv.) dropwise at −10° C. The reaction mixture was stirred for 1 h at −10° C. and quenched with 35% HCl. Organic phase was separated and washed with 35% HCl, the aqueous layer was extracted with DCM. The combined organic phase was washed with 20 wt % Na₂SO₄ and concentrated below 40° C. to give methyl (S)-(3-chloro-2-hydroxypropyl)carbamate (Formula 6).

Alternatively Formula 6 can be made by adding Enzyme (Novazym® 435) to a suspension of (S)-1-amino-3-chloropropan-2-ol hydrochloride in dimethyl carbamate (DMC), followed by diethylamine (DEA) dropwise at 35° C. for >2 h. The reaction mixture was stirred for 18 h at 35° C. and then filtered. The filtrate was concentrated to 9-11 vol. below 35° C. 6N HCl was charged into the organic phase and the mixture was stirred for 4-10 h. The aqueous phase was cut and the organic layer was concentrated below 35° C. to give methyl (S)-(3-chloro-2-hydroxypropyl)carbamate (Formula 6).

### Step 5

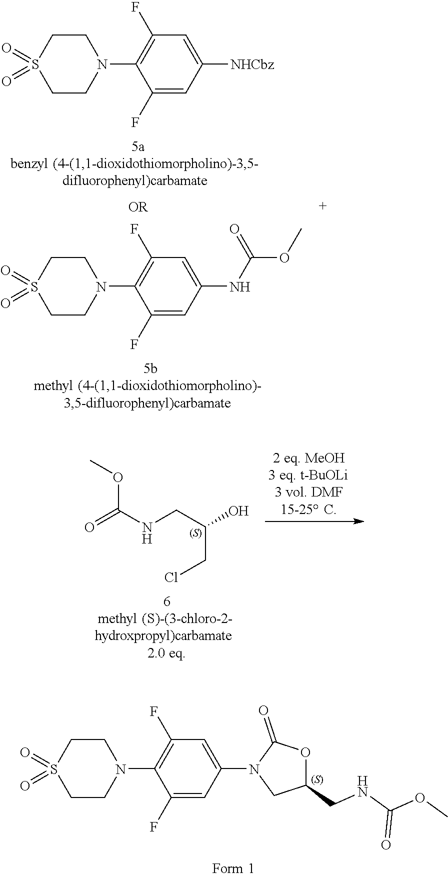

Lithium tert-butoxide (t-BuOLi) was added into a solution of benzyl (4-(1,1-dioxidothiomorpholino)-3,5-difluorophenyl)carbamate (5a) in N,N-dimethylformate (DMF) at 0° C., followed by methanol. A solution of methyl (S)-(3-chloro-2-hydroxypropyl)carbamate (6) in DMF was then added dropwise at 20° C. The resulting mixture was stirred at 20° C. for 24 h with product crystallization. The reaction was quenched with 1N HCl (aq.) until pH=4 at 5° C. and product further crystallized with addition of water. The resulting slurry was filtered and the cake was washed with water. The wet cake was dried via vacuum below 50° C. to give methyl ({(5S)-3-[4-(1,1-dioxo-1λ⁶-thiomorpholin-4-yl)-3,5-difluorophenyl]-2-oxo-1,3-oxazolidin-5-yl}methyl)carbamate (Formula I, as Form I) with 99.1% purity and 99.9% chiral purity.

Alternatively, Lithium tert-butoxide (t-BuOLi) was added into a solution of methyl (4-(1,1-dioxidothiomorpholino)-3,5-difluorophenyl)carbamate (5b) in N,N-dimethylformate (DMF) at 0° C. The mixture was warmed up to 20° C. when methanol was charged. Methyl (S)-(3-chloro-2-hydroxypropyl)carbamate (6) in DMF was added dropwise at 20° C. The resulting solution was stirred at 20° C. for 20-24 h, when the product gradually crystallized from reaction. At 5° C., the reaction was quenched with 1N HCl until ˜pH3-5 and then water was charged as an anti-solvent for product crystallization. The resulting slurry was filtered and the cake was washed with water. The wet cake was dried via vacuum below 50° C. to give methyl ({(5S)-3-[4-(1,1-dioxo-1λ⁶-thiomorpholin-4-yl)-3,5-difluorophenyl]-2-oxo-1,3-oxazolidin-5-yl}methyl)carbamate (Formula I, Form I) with 99.1% purity and 99.9% chiral purity.

### Step 6

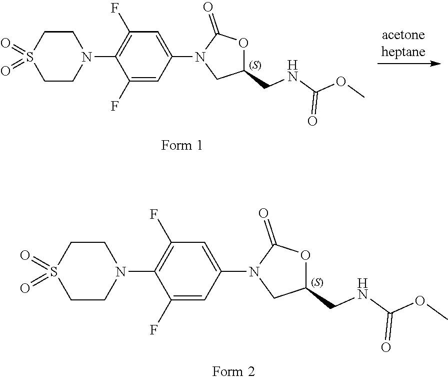

Formula I (Form I) was dissolved in acetone at 45 to 55° C. The batch was cooled to 40-50° C. and seeded with Formula I (Form II), aged and cooled to 20-30° C., then diluted with n-heptane. The slurry was further cooled to −5 to 5° C., aged, and then filtered. The wet cake was washed with the mixture solution of acetone and n-heptane, and then acetone. The wet cake was then dried to give solid Formula I (Form II) in 92% yield.

### Example 2

### Alternative Preparations of Form I

Alternatively, Form I can also be produced by solvent/anti solvent crystallizations where the solvent is (but limited not) DMSO or acetone and the antisolvent is (but not limited to) water, hexanes or heptanes. Form I can also be produced by slow evaporation of clear solutions of crude Example 1 in volatile solvents where the volatile solvent is (but not limited to) dichloromethane, acetone, or acetonitrile. Form I was characterized using PXRD, DSC, TGA and SCXRD. Form I is a kinetic phase and will recrystallize into Form II with time.

Form I was produced by crystallization of crude methyl ({(5S)-3-[4-(1,1-dioxo-1λ⁶-thiomorpholin-4-yl)-3,5-difluorophenyl]-2-oxo-1,3-oxazolidin-5-yl}methyl)carbamate in N,N-dimethylformamide/water as described in section Example 1. Form I was also produced by, in a 4 mL amber glass vial, dissolving 25 mg of crude Example 1 (25 mg), in 1.25 mL acetone with heat and under magnetic stirring. Water, 2 mL was added at once and stirring maintained for 15-30 min. The mixture was centrifuged, and Form I precipitate collected and dried under ambient conditions.

### Example 3

### Alternative Preparations of Form II

Form II can be produced by recrystallization of Form I in a variety of organic solvents or mixtures thereof where the solvent(s) is (are) (but not limited to) ethyl acetate, isopropanol, acetone, dichloromethane, acetonitrile, tetrahydrofuran, water, ethanol, 2-methyl tetrahydrofuran, toluene, methanol, dimethyl formamide or cyclopentyl methyl ether. Form II was characterized using PXRD, DSC and TGA. Form II is the most thermodynamically stable form in the 5-70° C. range.

Into a 4 mL amber glass vial equipped with a magnetic stir bar, about 100 mg of Form I was suspended in 1 mL of acetone. The mixture was stirred at 25° C. for 14 days and the resulting precipitate was isolated by centrifugation. The Form II crystalline solid was allowed to dry in air before storage and further use.

Alternatively, about 32 kg of crude Example 1 was dissolved in 321 kg of acetone into an appropriate vessel at 45-55° C. The solution was then filtered into a clean vessel and kept warm. The vessel temperature was then adjusted to 40 to 50° C. before 98 g of Form II seeds were added. The mixture was stirred at 40 to 50° C. for 5 h before it is cooled down to 20 to 30° C. in 8-11 h. About 648 Kg of n-heptane was then added dropwise at 20 to 30° C. and the mixture cooled down to −5 to 5° C. in 6 h. The mixture was stirred at −5 to 5° C. for 1 to 3 h and filtered. The resulting product was further washed with acetone/n-heptane (1/2, V/V) and neat acetone before it was dried at 40 to 50° C. under reduced pressure for 12 h yielding crystalline Form II.

### Example 4

### Preparation of Type C

Type C can be produced by recrystallization of either Form I, Form II or Form III in solvent/antisolvent systems where the solvent is (but not limited to) acetone or dichloromethane and the antisolvent is (but not limited to) n-hexane or n-heptane. Type C is highly metastable and has a few hours lifetime in solution before turnovers into a more thermodynamically stable phase (Form I, Form II or Form III).

Into a 50 mL Schott bottle, 300 mg of Form II was dissolved in 13.5 mL THF at 55° C. The solution was then moved to 25° C. and a magnetic stir bar was inserted. n-hexane, 30 mL was added at once and mild stirring (300 rpm) was applied and maintained 1 h at room temperature. The crystalline suspension was then filtered and dried under vacuum at 25° C. yielding Type C.

### Example 5

### Preparation of Form III

Form III was produced by recrystallization of Type C in the solid state at 150° C. and under a N₂ flow. Form III can also be formed in some instances from solution in acetone/n-heptane crystallizations. Quick turnovers to more thermodynamically stable phases Form I or Form II are readily observed within minutes to hours. Form III was characterized using PXRD, DSC, TGA and SCXRD.

About 15 mg of Type C was loaded into an aluminum DSC pan. The open DSC pan was heated to 150° C. at 10° C./min (under a 50 mL/min N₂ flow), held for 5 min at 150° C. and cooled down to 25° C. at 10° C./min. The resulting Form III solid in the DSC pan was then scratched off and collected.

### PXRD

As those of ordinary skill in the art readily appreciate, the physical characteristics of a crystal may be effectively characterized by powder x-ray diffraction (PXRD) analysis. Such characterizations may be used to distinguish such crystals from other crystals. For each of the crystalline forms of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate described herein, PXRD analysis was completed on a dry cake sample. PXRD data reported herein were acquired on a Bruker D8 Advance System configured in the Bragg-Brentano configuration and equipped with a Cu radiation source with monochromatization to Kα achieved using a nickel filter. A fixed slit optical configuration was employed for data acquisition. Data were acquired between 3 and 40° 2θ and a step size of 0.018°. Samples were prepared by gently pressing the samples onto a shallow cavity zero background silicon holder.

Those skilled in the art will recognize that the measurements of the PXRD peak locations for a given crystalline form of the same compound will vary within a margin of error. The margin of error for the 2-theta values measured as described herein is typically +/−0.2° 2θ. Variability can depend on such factors as the system, methodology, sample, and conditions used for measurement. As will also be appreciated by the skilled crystallographer, the intensities of the various peaks reported in the figures herein may vary due to several factors such as orientation effects of crystals in the x-ray beam, the purity of the material being analyzed, and/or the degree of crystallinity of the sample. The skilled crystallographer also will appreciate that measurements using a different wavelength will result in different shifts according to the Bragg-Brentano equation. Such further PXRD patterns generated by use of alternative wavelengths are alternative representations of the PXRD patterns of the crystalline material of the invention and as such are within the scope of the invention.

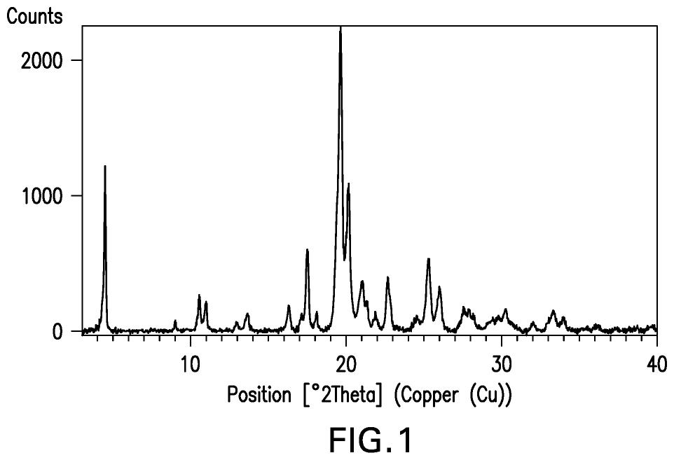

A PXRD pattern of crystalline Form 1, generated using the equipment and procedures described above, is displayed in FIG. 1. A crystalline Form I characterized by a powder x-ray diffraction pattern is shown in FIG. 1.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 1 (+/−0.2° 2 theta). The locations of these PXRD peaks are characteristic of the crystalline Form I. Thus, in another aspect, crystalline Form I is characterized by a powder x-ray diffraction pattern having each of the peak positions listed in Table 1, +/−0.2° 2-theta.

TABLE 1
Diffraction peaks and corresponding
d-spacings for experimental Form I

| Pos. [° Two Theta] | d-spacing [Å] | Rel. Int. [%] |
|---|---|---|
| 4.49 | 19.69 | 54.69 |
| 9.00 | 9.83 | 3.52 |
| 10.55 | 8.39 | 12.08 |
| 10.98 | 8.06 | 10.66 |
| 13.00 | 6.81 | 2.93 |
| 13.65 | 6.49 | 6.42 |
| 16.31 | 5.44 | 9.23 |
| 17.14 | 5.17 | 6.60 |
| 17.50 | 5.07 | 27.80 |
| 18.10 | 4.90 | 7.26 |
| 19.64 | 4.52 | 100.00 |
| 20.14 | 4.41 | 48.95 |
| 21.03 | 4.22 | 16.93 |
| 21.32 | 4.17 | 10.66 |
| 21.90 | 4.06 | 6.62 |
| 22.68 | 3.92 | 18.50 |
| 24.55 | 3.63 | 5.63 |
| 25.29 | 3.52 | 25.03 |
| 26.00 | 3.43 | 15.34 |

In a further aspect, the PXRD peak locations displayed in Table 1 and/or FIG. 1 most characteristic of crystalline Form I can be selected and grouped as “diagnostic peak set” to conveniently distinguish this crystalline form from others. A diagnostic peak is defined when no other diffraction peak belonging to another crystalline phase exists within ±0.2° 2Theta. Selections of such characteristic peaks are set out in Table 2.

TABLE 2
Diagnostic peak set selected for experimental Form I

| Pos. [° Two Theta] | d-spacing [Å] | Rel. Int. [%] |
|---|---|---|
| 10.55 | 8.39 | 12.08 |
| 13.65 | 6.49 | 6.42 |

Thus, in another aspect, there is provided a crystalline Form I characterized by a powder X-ray diffraction pattern comprising each of the 2-theta values listed in Diagnostic Peak Set in Table 2, +/−0.2° 2-theta.

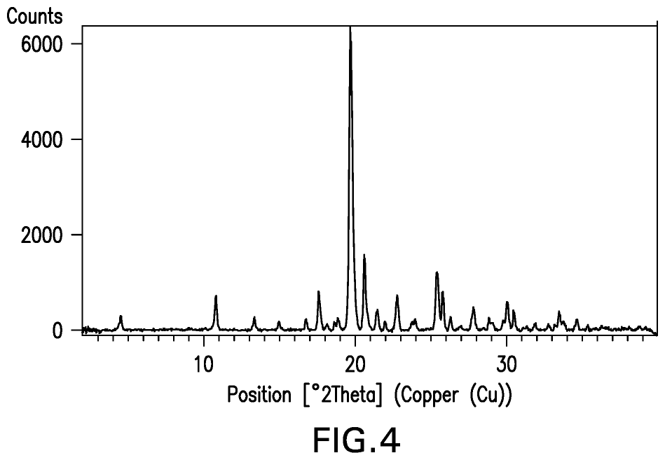

A PXRD pattern of crystalline Form II, generated using the equipment and procedures described above, is displayed in FIG. 4. A crystalline Form II characterized by a powder x-ray diffraction pattern is shown in FIG. 4.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 3 (+/−0.2° 2 theta). The locations of these PXRD peaks are characteristic of crystalline Form II. Thus, in another aspect, crystalline Form II is characterized by a powder x-ray diffraction pattern having each of the peak positions listed in Table 3, +/−0.2° 2-theta.

TABLE 3
Diffraction peaks and corresponding
d-spacings for experimental Form II

| Pos. [° Two Theta] | d-spacing [Å] | Rel. Int. [%] |
|---|---|---|
| 4.53 | 19.51 | 5.75 |
| 10.81 | 8.19 | 12.02 |
| 13.35 | 6.63 | 5.07 |
| 14.97 | 5.92 | 3.94 |
| 16.76 | 5.29 | 4.76 |
| 17.62 | 5.04 | 12.55 |
| 18.14 | 4.89 | 3.19 |
| 18.65 | 4.76 | 3.64 |
| 18.88 | 4.70 | 4.76 |
| 19.70 | 4.51 | 100.00 |
| 20.62 | 4.31 | 24.61 |
| 21.47 | 4.14 | 7.82 |
| 22.00 | 4.04 | 3.72 |
| 22.79 | 3.90 | 12.36 |
| 23.76 | 3.74 | 3.80 |
| 23.97 | 3.71 | 4.41 |
| 25.42 | 3.50 | 19.55 |
| 25.79 | 3.45 | 13.29 |
| 26.30 | 3.39 | 5.28 |
| 27.81 | 3.21 | 8.51 |
| 28.86 | 3.09 | 5.05 |
| 30.05 | 2.97 | 10.66 |

In a further aspect, the PXRD peak locations displayed in Table 3 and/or FIG. 4 most characteristic of crystalline Form II can be selected and grouped as “diagnostic peak set” to conveniently distinguish this crystalline form from others. A diagnostic peak is defined when no other diffraction peak belonging to another crystalline phase exists within ±0.2° 2Theta. Selections of such characteristic peaks are set out in Table 4.

TABLE 4
Diagnostic peak set selected for experimental Form II

| Pos. [° Two Theta] | d-spacing [Å] | Rel. Int. [%] |
|---|---|---|
| 13.35 | 6.63 | 5.07 |
| 14.97 | 5.92 | 3.94 |
| 20.62 | 4.31 | 24.61 |
| 28.86 | 3.09 | 5.05 |
| 30.05 | 2.97 | 10.66 |

Thus, in another aspect, there is provided a crystalline Form II characterized by a powder x-ray diffraction pattern comprising each of the 2-theta values listed in Diagnostic Peak Set in Table 4, +/−0.2° 2-theta.

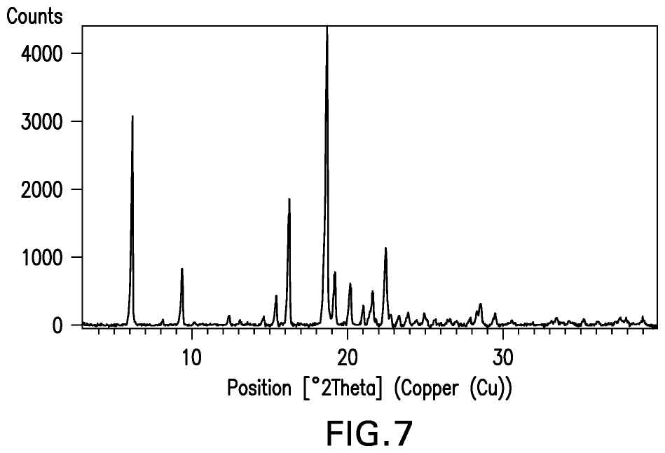

A PXRD pattern of crystalline Type C generated using the equipment and procedures described above is displayed in FIG. 7. Thus, in another aspect, there is provided a crystalline Type C characterized by a powder X-ray diffraction pattern substantially as shown in FIG. 7.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 5 (+/−0.2° 2 theta). The locations of these PXRD peaks are characteristic of the crystalline Type C. Thus, in another aspect, crystalline Type C is characterized by a powder x-ray diffraction pattern having each of the peak positions listed in Table 5, +/−0.2° 2-theta.

TABLE 5
Diffraction peaks and corresponding d-spacings
for experimental crystalline Type C

| Pos. [° Two Theta] | d-spacing [Å] | Rel. Int. [%] |
|---|---|---|
| 6.19 | 14.28 | 87.69 |
| 8.15 | 10.84 | 1.86 |
| 9.41 | 9.40 | 18.83 |
| 10.19 | 8.68 | 1.72 |
| 12.44 | 7.12 | 4.54 |
| 13.12 | 6.75 | 1.80 |
| 14.68 | 6.03 | 2.07 |
| 15.44 | 5.74 | 10.71 |
| 16.28 | 5.45 | 46.90 |
| 18.69 | 4.75 | 100.00 |
| 19.22 | 4.62 | 19.65 |
| 20.21 | 4.39 | 14.63 |
| 21.06 | 4.22 | 7.38 |
| 21.65 | 4.10 | 12.86 |
| 22.49 | 3.95 | 24.65 |
| 22.84 | 3.89 | 5.33 |
| 23.34 | 3.81 | 4.26 |
| 23.93 | 3.72 | 4.61 |
| 24.46 | 3.64 | 2.01 |
| 24.99 | 3.56 | 4.52 |

In a further aspect, the PXRD peak locations displayed in Table 5 and/or FIG. 7 most characteristic of crystalline Type C can be selected and grouped as “diagnostic peak set” to conveniently distinguish this crystalline form from others. A diagnostic peak is defined when no other diffraction peak belonging to another crystalline phase exists within ±0.2° 2Theta. Selections of such characteristic peaks are set out in Table 6.

TABLE 6
Diagnostic peak set selected for experimental crystalline Type C

| Pos. [° Two Theta] | d-spacing [Å] | Rel. Int. [%] |
|---|---|---|
| 6.19 | 14.28 | 87.69 |
| 8.15 | 10.84 | 1.86 |
| 9.41 | 9.40 | 18.83 |
| 10.19 | 8.68 | 1.72 |
| 12.44 | 7.12 | 4.54 |
| 14.68 | 6.03 | 2.07 |
| 19.22 | 4.62 | 19.65 |
| 24.99 | 3.56 | 4.52 |

Thus, in another aspect, there is provided a crystalline Type C characterized by a powder x-ray diffraction pattern comprising each of the 2-theta values listed in Diagnostic Peak Set in Table 6, +/−0.2° 2-theta.

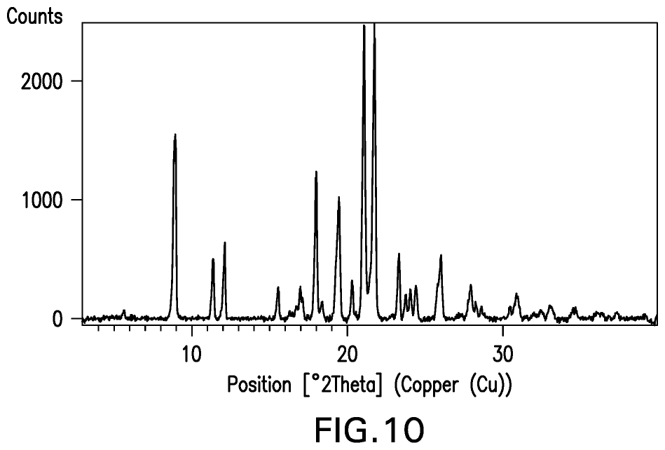

A PXRD pattern of crystalline Form III generated using the equipment and procedures described above is displayed in FIG. 10. Thus, in another aspect, there is provided a crystalline Form III characterized by a powder x-ray diffraction pattern substantially as shown in FIG. 10.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 7 (+/−0.2° 2 theta). The locations of these PXRD peaks are characteristic of the crystalline Form III. Thus, in another aspect, crystalline Form III is characterized by a powder x-ray diffraction pattern having each of the peak positions listed in Table 7, +/−0.2° 2-theta.

TABLE 7
Diffraction peaks and corresponding d-spacings
for experimental crystalline Form III

| Pos. [° Two Theta] | d-spacing [Å] | Rel. Int. [%] |
|---|---|---|
| 5.72 | 15.44 | 3.53 |
| 8.99 | 9.83 | 67.92 |
| 11.44 | 7.73 | 21.80 |
| 12.19 | 7.26 | 27.45 |
| 15.63 | 5.67 | 11.85 |
| 16.36 | 5.42 | 3.35 |
| 16.56 | 5.35 | 3.01 |
| 16.83 | 5.27 | 5.53 |
| 17.05 | 5.20 | 12.01 |
| 17.19 | 5.16 | 8.56 |
| 18.07 | 4.91 | 51.95 |
| 18.46 | 4.81 | 7.15 |
| 19.52 | 4.55 | 41.29 |
| 20.40 | 4.35 | 14.13 |
| 21.15 | 4.20 | 98.04 |
| 21.82 | 4.07 | 100.00 |
| 23.40 | 3.80 | 22.02 |
| 23.83 | 3.73 | 9.03 |
| 24.14 | 3.69 | 11.26 |
| 24.49 | 3.63 | 12.17 |
| 26.10 | 3.41 | 22.36 |
| 28.02 | 3.19 | 12.75 |

In a further aspect, the PXRD peak locations displayed in Table 7 and/or FIG. 10 most characteristic of crystalline Form III can be selected and grouped as “diagnostic peak set” to conveniently distinguish this crystalline form from others. A diagnostic peak is defined when no other diffraction peak belonging to another crystalline phase exists within ±0.2° 2Theta. Selections of such characteristic peaks are set out in Table 8.

TABLE 8
Diagnostic peak set selected for experimental crystalline Form III

| Pos. [° Two Theta] | d-spacing [Å] | Rel. Int. [%] |
|---|---|---|
| 5.72 | 15.44 | 3.53 |
| 11.44 | 7.73 | 21.80 |
| 12.19 | 7.26 | 27.45 |

Thus, in another aspect, there is provided a crystalline Form III characterized by a powder x-ray diffraction pattern comprising each of the 2-theta values listed in Diagnostic Peak Set in Table 8, ±/−0.2° 2-theta.

### Single Crystal Crystallographic Information

Single crystal structure determination was performed using a Bruker APEX 2 CCD diffractometer. Cell determinations and diffraction data were measured using monochromatized Cu Kα radiation. Full data acquisition for structure solution was performed at 100K with unit cell dimensions also acquired at 295K.

The crystal structure of Form I was solved by single crystal X-ray analysis. Crystallographic details are shown in Table 9.

TABLE 9
Crystallographic Information for Form I

| Formula, Formula weight | C16H19N3O6F2S, 419.40 |
|---|---|
| Crystal system, Space group | Triclinic, P1 |
| Cell lengths | a = 5.048 (10), b = 8.921 (15), |
| | c = 19.60 (3) |
| Cell angles | α = 86.39(3), β = 87.08(3), |
| | γ = 85.30(6) |
| V(Å³), Z, Z′, D(calc) | 877(3), 2, 2, 1.589 |
| μ(Cu Kα) (mm⁻¹) | 2.226 |
| F(000) | 436 |
| Crystal size (mm) | 0.12 × 0.06 × 0.02 |
| Temperature (K) | 100 |
| Radiation (Å) | Cu Kα (1.54184) |
| Instrument | Bruker APEXII |
| Resolution (Å⁻³), max theta (°) | 0.84, 70.829 |
| Reflections: (Total, Unique, | 18885, 6410, 5997 |
| 2σ Obsd) | |
| Refined parameters | 507 |
| R, wR₂, S | 0.035, 0.085, 1.031 |
| Max. shift/error | 0.000 |
| Max. residual density [e Å⁻³] | 0.27 |

The unit cell dimensions for Form I acquired at 295K are a=5.0723 (17), b=9.065 (5), c=19.639 (17), α=86.73 (5), β=87.37 (5), γ=84.94 (4).

The crystal structure of Form III was solved by single crystal X-ray analysis. Crystallographic details are shown in Table 10.

TABLE 10
Crystallographic Information for Form III

| Formula, Formula weight | C16H19N306F2S, 419.40 |
|---|---|
| Crystal system, Space group | Triclinic, P1 |
| Cell lengths | a = 5.7320 (2), b = 10.6688 |
| | (4), c = 15.9437 (6) |
| Cell angles | α = 72.0780 (10), β = 87.6970 |
| | (10), γ = 77.1800 (10) |
| V(Å³), Z, Z′, D(calc) | 904.16(6), 2, 2, 1.541 |
| μ(Cu Kα) (mm⁻¹) | 2.159 |
| F(000) | 436 |
| Crystal size (mm) | 0.25 × 0.13 × 0.05 |
| Temperature (K) | 100 |
| Radiation (Å) | Cu Kα (1.54184) |
| Instrument | Bruker APEXII |
| Resolution (Å⁻³), max theta (°) | 0.84, 66.652 |
| Reflections: (Total, Unique, | 20123, 6137, 6018 |
| 2σ Obsd) | |
| Refined parameters | 507 |
| R, wR₂, S | 0.030, 0.083, 1.058 |
| Max. shift/error | 0.000 |
| Max. residual density [e Å⁻³] | 0.39 |

The unit cell dimensions or Type C acquire at 295K are a=5.7897 (4), =10.6620 (16), c=16.246 (3), α=72.028 (12), β=86.739 (8), γ=77.881 (9).

### TGA

Thermogravimetric analyses (TGA) were performed on a TA instrument model Discovery TGA 5500. Experiments were performed under a 25 mL/min flow of nitrogen and using a heating rate of 10° C./min to a maximum temperature of approximately 250° C. About 5-10 mg of sample was loaded into a 50 uL aluminum pan and loaded onto the instrument before the measurement method was started. Data analyses were performed on the TRIOS software by TA. Weight losses are reported up to ca. 150° C., temperature at which most organic solvents evaporate. Decomposition of the materials are not reported but are seen from ca. 200° C. for all claimed crystalline forms.

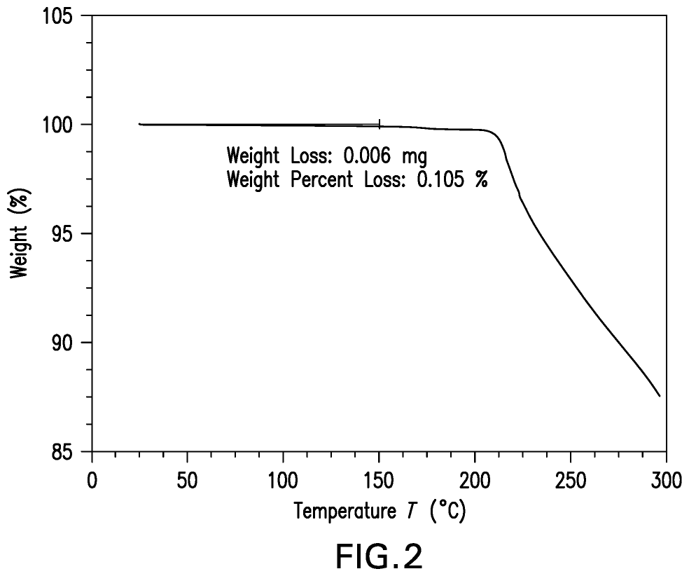

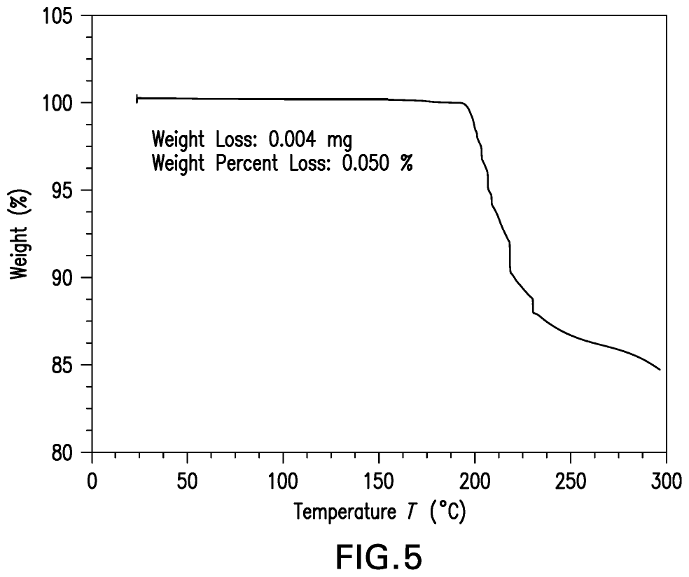

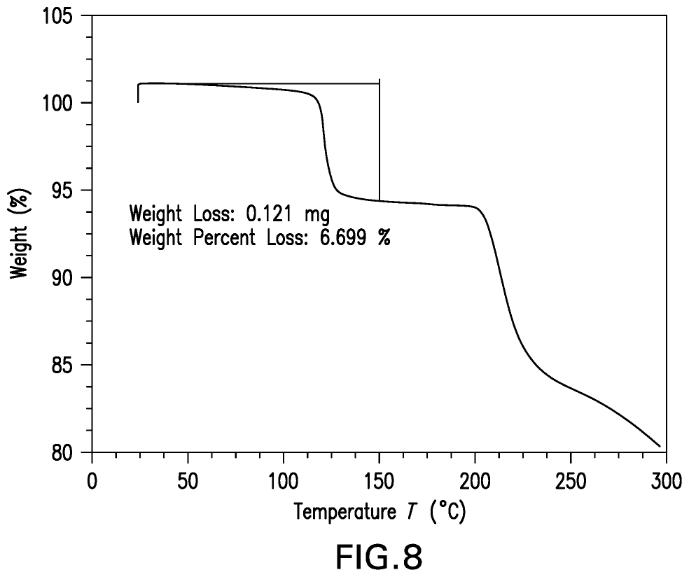

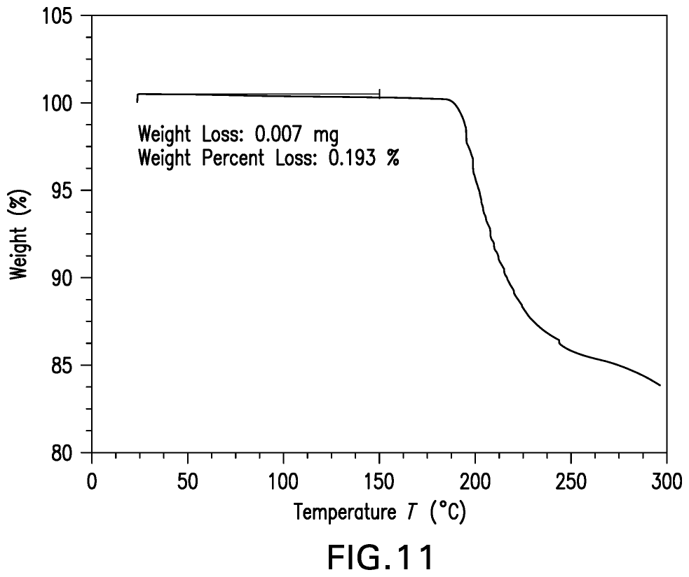

Crystalline Form I can be further characterized by the TGA curve of FIG. 2. Crystalline Form II can be further characterized by the TGA curve of FIG. 5. Crystalline Type C can be further characterized by the TGA curve of FIG. 8. Crystalline Form III can be further characterized by the TGA curve of FIG. 11.

### DSC

Differential Scanning Calorimetry data were acquired using a TA instrument model Discovery DSC 2500. Experiments were performed under a 50 mL/min flow of nitrogen and a heating rate of 10° C./min up to a maximum temperature of approximately 250° C. About 5-10 mg of sample was loaded into a T-zero hermetic pan and sealed with a hermetic lid. Two pin holes were made on the sealed lid to allow residual water and organics release throughout the measurement. Data analyses were performed using the TRIOS software provided by TA. The data reported are the onset temperature, peak temperature, and enthalpy.

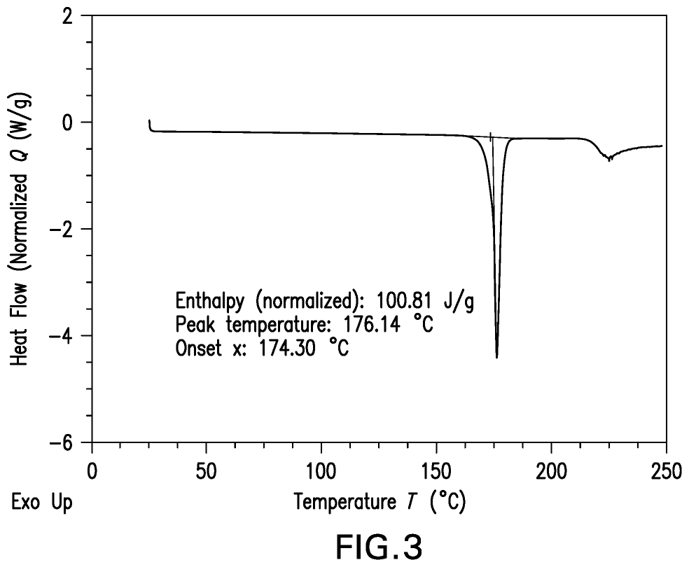

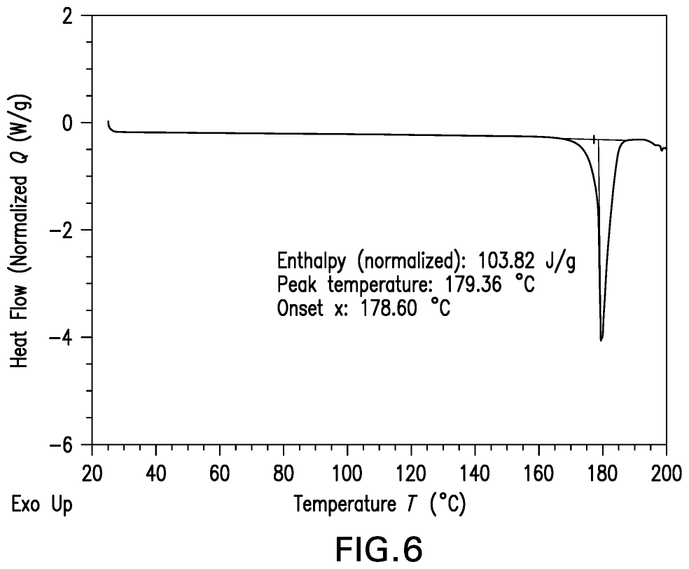

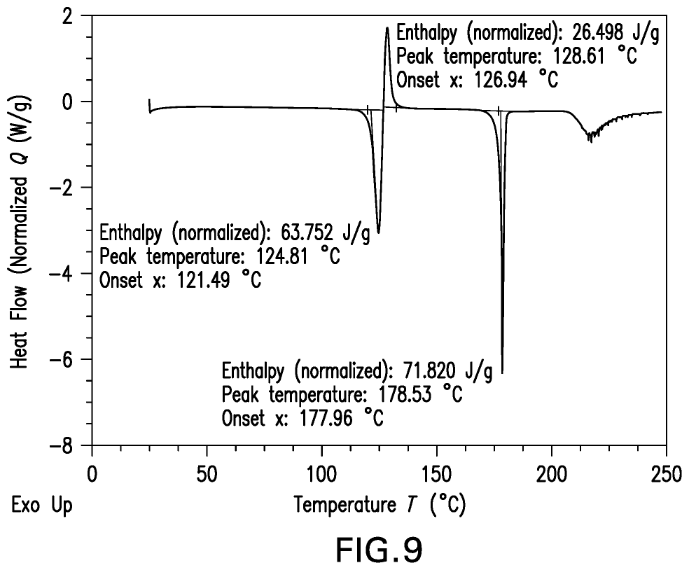

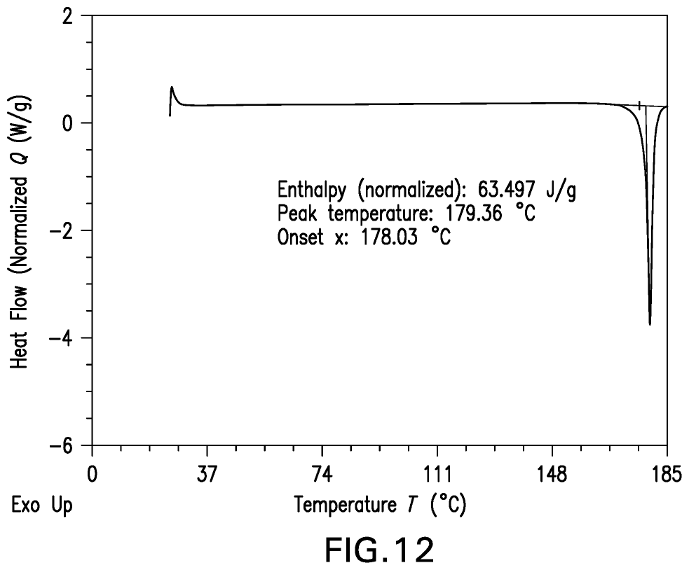

Crystalline Form I of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can be further characterized by the DSC curve of FIG. 3. Crystalline Form II of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can be further characterized by the DSC curve of FIG. 6. Crystalline Type C of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can be further characterized by the DSC curve of FIG. 9. Crystalline Form III of methyl ({(5S)-3-[4-(1,1-dioxo-1λ⁶-thiomorpholin-4-yl)-3,5-difluorophenyl]-2-oxo-1,3-oxazolidin-5-yl}methyl)carbamate can be further characterized by the DSC curve of FIG. 12.

A representative sample of Form I was analyzed by TGA and DSC according to the methods described above. Form I displays one single melting endotherm with Tonset=174.30° C., Tpeak=176.14° C., and ΔH=100.81 J/g (FIG. 3). Thermogravimetric analysis exhibits a negligible weight loss of 0.10% between room temperature and 150° C. (FIG. 2.).

A representative sample of Form II was analyzed by TGA and DSC according to the methods described above. Form II displays one single melting endotherm with Tonset=178.60° C., Tpeak=179.36° C., and ΔH=103.82 J/g (FIG. 6). Thermogravimetric analysis exhibits an unsignificant weight loss of 0.05% between room temperature and 150° C. (FIG. 5.).

A representative sample of Type C was analyzed by TGA and DSC according to the methods described above. Type C displays (1) an endotherm attributed to the evaporation of water and the melting of the solid with Tonset=121.49° C., Tpeak=124.81° C., and ΔH=63.75 J/g, (2) a recrystallization exotherm with Tonset=126.94° C., Tpeak=128.61° C., and ΔH=26.50 J/g and (3) a melting endotherm with Tonset=177.96° C., Tpeak=178.53° C., and ΔH=71.82 J/g (FIG. 9.). Thermogravimetric analysis exhibits a weight loss of 6.70% between room temperature and ca. 150° C. attributed to the evaporation of water (FIG. 8). The total absence of crystallization solvents (solvate formation) in the Type C solid was further confirmed through gas chromatography analysis.

A representative sample of Form III was analyzed by TGA and DSC according to the methods described above. Form III displays a melting endotherm with Tonset=178.03° C., Tpeak=179.36° C., and ΔH=63.50 J/g (FIG. 12.). Thermogravimetric analysis exhibits an unsignificant weight loss of 0.19% between room temperature and 150° C. (FIG. 11).

### Example 6

Three different strengths (10 mg, 100 mg, and 300 mg) capsules were made using crystalline Form II. The three formulations are shown in Tables 11-13 below. For the manufacturing process, sieved Form II, microcrystalline cellulose, lactose monohydrate, croscarmellose sodium and colloidal silicon dioxide were roller compacted or blended and/or co-milled and/or blended again. Sieved magnesium stearate was added and the mixture was encapsulated using Size 0 Capsules. The capsules were polished, ran through a metal detector and weight sorted.

TABLE 11

| Material Description | Amount (mg) |
|---|---|
| Form II | 10.0 |
| Microcrystalline Cellulose (Avicel ® PH 102) | 28.6 |
| Lactose Monohydrate Sheffield ™ Spray Dried | 114.2 |
| Fast Flo 316 | |
| Croscarmellose Sodium (Ac-Di-Sol ® SD-711) | 4.8 |
| Colloidal Silicon Dioxide (Cab-O-Sil ® M5P) | 0.8 |
| Magnesium Stearate (Hy qual 2257) | 1.6 |
| Capsules, HPMC, Size 3, White/White, Opaque, | 46.0 |
| Unmarked, Vcaps | |

TABLE 12

| Material Description | Amount (mg) |
|---|---|
| Form II | 100.0 |
| Microcrystalline Cellulose (Avicel ® PH 102) | 56.4 |
| Lactose Monohydrate Sheffield ™ Spray Dried | 225.6 |
| Fast Flo 316 | |
| Croscarmellose Sodium (Ac-Di-Sol ® SD-711) | 12.0 |
| Colloidal Silicon Dioxide (Cab-O-Sil ® M5P) | 2.0 |
| Magnesium Stearate (Hyqual 2257) | 4.0 |
| Capsules, HPMC, Size 3, White/White, Opaque, | 93.0 |
| Unmarked, Vcaps | |

TABLE 13

| Material Description | Amount (mg) |
|---|---|
| Form II | 300.0 |
| Microcrystalline Cellulose (Avicel ® PH 102) | 44.5 |
| Lactose Monohydrate Sheffield ™ Spray Dried | 178.0 |
| Fast Flo 316 | |
| Croscarmellose Sodium (Ac-Di-Sol ® SD-711) | 16.505 |
| Colloidal Silicon Dioxide (Cab-O-Sil ® M5P) | 5.5 |
| Magnesium Stearate (Hyqual 2257) | 5.5 |
| Capsules, HPMC, Size 3, White/White, Opaque, | 115.0 |
| Unmarked, Vcaps | |

### Example 7

Amorphous dispersions of the compound of Formula I stabilized with polymers were prepared. Methyl ({(5S)-3-[4-(1,1-dioxo-1λ6-thiomorpholin-4-yl)-3,5-difluorophenyl]-2-oxo-1,3-oxazolidin-5-yl}methyl)carbamate/polymer miscibility studies with film cast amorphous solid dispersions and cryomilled blends were performed. Two drug-polymer systems, one using vinyl pyrrolidone-vinyl acetate (PVP VA64) and another hydroxypropyl methyl cellulose acetate succinate (HPMCAS-MF), with 25% drug loading were selected for further formulation development.

Hot melt extrusion (HME) processes were developed for drug intermediate preparation and a Direct Encapsulation-Dry Filled Capsule (DE-DFC) process was carried out for prototype capsule filling. Lactose SD FAST FLO 316, Microcrystalline Cellulose PH-102, Croscarmellose Sodium SD-711 and Magnesium Stearate 5712 were used along with Size 00 HPMC capsules were used to make 100 mg capsules of methyl ({(5S)-3-[4-(1,1-dioxo-1λ6-thiomorpholin-4-yl)-3,5-difluorophenyl]-2-oxo-1,3-oxazolidin-5-yl}methyl)carbamate per capsule. The HME intermediate and capsule formulations of PVP VA64 based are listed in Table 14 and Table 15. The formulations of HPMCAS-MF based are listed in Table 16 and Table 17.

TABLE 14
Formulation of HME Intermediate PVP VA64 Based

| | HME intermediate | | |
|---|---|---|---|
| | PVP VA64 based | | |
| | | Composition | Quantity/ |
| Material name | Function | % | g |
| methyl ({(5S)-3-[4-(1,1-dioxo-1λ⁶- | API | 25.00 | 45.0 |
| thiomorpholin-4-yl)-3,5- | | | |
| difluorophenyl]-2-oxo-1,3- | | | |
| oxazolidin-5-yl}methyl)carbamate | | | |
| Polyvinylpyrrolidone/vinyl Acetate | Polymer | 75.00 | 135.0 |
| Copolymer Kollidon VA64 | | | |

| | | |
|---|---|---|
| Total | 100.0 | 180.0 |

TABLE 15
Formulation of 100 mg Capsules PVP VA64 Based

| | 100 mg capsules PVP VA64 based | | | |
|---|---|---|---|---|
| Material name | Function | Composition % | Unit weight/mg | Quantity/g |
| HME intermediate PVP VA64 based | Active intermediate | 60.00 | 400.0 | 126.51 |
| Lactose SD FAST FLO 316 | Brittle filler | 28.80 | 192.0 | 60.72 |
| Microcrystalline Cellulose PH-102 | Plastic filler | 7.200 | 48.00 | 15.18 |
| Croscarmellose Sodium SD-711 | Disintegrant | 3.000 | 20.00 | 6.33 |
| Magnesium Stearate 5712 | Lubricant | 1.000 | 6.700 | 2.11 |

| | | | |
|---|---|---|---|
| Total | 100.0 | 666.7 | 210.85 |

TABLE 16
Formulation of HME Intermediate HPMCAS-MF Based

| | HME intermediate | | |
|---|---|---|---|
| | HPMCAS-MF based | | |
| | | Composition | Quantity/ |
| Material name | Function | % | g |
| methyl ({(5S)-3-[4-(1,1-dioxo-1λ⁶- | API | 25.00 | 45.00 |
| thiomorpholin-4-yl)-3,5- | | | |
| difluorophenyl]-2-oxo-1,3- | | | |
| oxazolidin-5-yl}methyl)carbamate | | | |
| Hypromellose Acetate Succinate | Polymer | 75.00 | 135.00 |
| (Shin-Etsu AQOAT) AS-MF | | | |

| | | |
|---|---|---|
| Total | 100.0 | 180.00 |

TABLE 17
Formulation of 100 mg Capsules HPMCAS-MF Based

| | 100 mg capsules HPMCAS-MF based | | | |
|---|---|---|---|---|
| Material name | Function | Composition % | Unit weight/mg | Quantity/g |
| HME intermediate HPMCAS- MF based | Active | 60.00 | 400.0 | 123.41 |
| Lactose SD FAST FLO 316 | Brittle filler | 28.80 | 192.0 | 59.24 |
| Microcrystalline Cellulose PH-102 | Plastic filler | 7.200 | 48.00 | 14.81 |
| Croscarmellose Sodium SD-711 | Disintegrant | 3.000 | 20.00 | 6.17 |
| Magnesium Stearate 5712 | Lubricant | 1.000 | 6.700 | 2.06 |

| | | | |
|---|---|---|---|
| Total | 100.0 | 666.7 | 205.69 |

The API was sieved through 30 mesh screen and then sandwiched between the polymer. A 1 L container was used for PVP VA64 based blend, and 2 L container was used for HPMCAS-MF. The polymer and API were blended for 6 min 30 s at 46 RPM. For the hot melt extrusion, Thermo Fisher Pharma Mini Hot Melt Extruder was used. According to the appearance of extrudate under different heating temperatures, the heating temperature of 175° C. for the PVP VA64 extrusion and the heating temperature of 185° C. for the HPMCAS-MF extrusion was selected. The remaining blend of API and polymer was added into the hopper and extruded. FitzMill was used to mill the obtained extrudate. Knife impeller was used, and the impeller speed was set at 6000 RPM. Two times milling were performed. The extrudates were milled through 1.0 mm screen to get the coarse extrudates. Then the coarse extrudates were milled through 0.5 mm screen to obtain the fine HME intermediate powder.

The batch size of capsule filling was determined by the weight of remaining HME intermediate powder. The lactose and MCC PH-102 were sieved through 30 mesh screen separately. The magnesium stearate was sieved through 60 mesh screen. The HME intermediate powder was sandwiched between the lactose and MCC PH-102 in 1 L container and blended for 6 min 30 s at 46 RPM. Then the magnesium stearate was added into the container and lubrication for 2 min 10 s at 46 RPM.

A semi-automatic capsule filling machine was used for capsule filling. The target filling weight is 666.7 mg. The average weight of empty size 00 capsule shell is 118.3 mg. The individual capsule weight limit is 735.0-835.0 mg (±7.5%), the average 10 capsules weight limit is 751.7-818.3 mg (±5.0%). Weight sorting was performed after capsule filling. 18 EA capsules with one 1 g desiccant were filled into one 75 CC HDPE bottle and screwed the cap tightly. Sealed the bottles one by one using Induction Aluminum Foil sealer and performed full inspection.

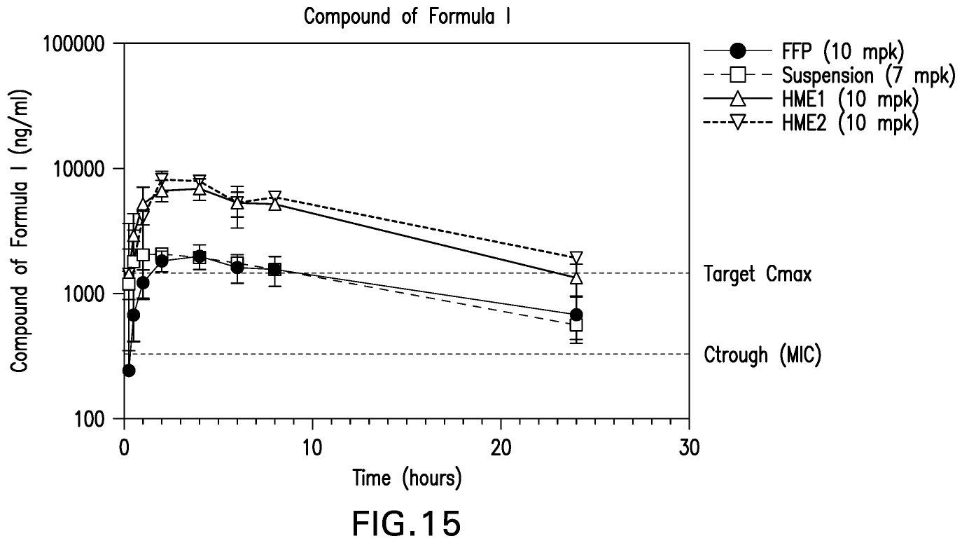

FIG. 15 shows the results of a dog PK study comparing the two amorphous hot melt extrusion formulations above, a suspension and capsule formulations of Example 7 with crystalline Form II. The results are also shown in Table 15. As shown in FIG. 15 and Table 18 the HME amorphous formulations are more bioavailable.

TABLE 18

| | Dose | Tmax | Cmax | Auc0-inf | Bioavailability |
|---|---|---|---|---|---|
| | (mg) | (h) | (ng/ml) | (ng/ml · h) | (F) |
| Form II Suspension | 70 | 2.3 | 2190 | 42718 | 63 |
| Form II Capsule | 100 | 4.3 | 2040 | 42430 | 44 |
| HME PVP VA64 | 100 | 3.3 | 7030 | 115020 | 120 |
| HME HPMCAS-MF | 100 | 3.3 | 8310 | 145820 | 152 |

1. A method for preparing a compound of Formula I 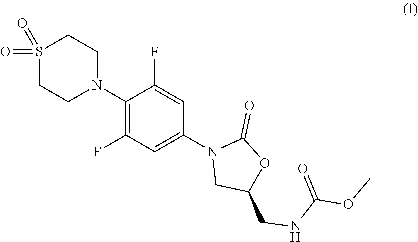 [SMILES: COC(=O)NC[C@H]1CN(c2cc(F)c(N3CCS(=O)(=O)CC3)c(F)c2)C(=O)O1]
or a pharmaceutically acceptable salt thereof, comprising treating a compound of Formula 5a 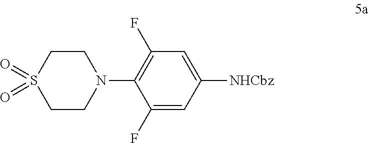 [SMILES: O=C(Nc1cc(F)c(N2CCS(=O)(=O)CC2)c(F)c1)OCc1ccccc1]
with methyl (S)-(3-chloro-2-hydroxypropyl)carbamate.

2. The method of claim 1 wherein the process of preparing a compound of Formula I 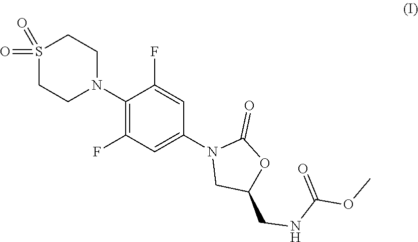 [SMILES: COC(=O)NC[C@H]1CN(c2cc(F)c(N3CCS(=O)(=O)CC3)c(F)c2)C(=O)O1]
or a pharmaceutically acceptable salt thereof, comprises treating a compound of Formula 5a 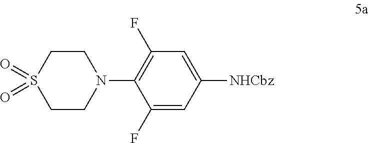 [SMILES: O=C(Nc1cc(F)c(N2CCS(=O)(=O)CC2)c(F)c1)OCc1ccccc1]
with methyl (S)-(3-chloro-2-hydroxypropyl)carbamate in the presence of a base.

3. The method of claim 2, wherein the base is selected from the group consisting of sodium methoxide, potassium methoxide, lithium methoxide, sodium t-butoxide, potassium t-butoxide and lithium t-butoxide.

4. The method of claim 2, wherein the base is lithium t-butoxide.

5. A method for preparing a compound of Formula I 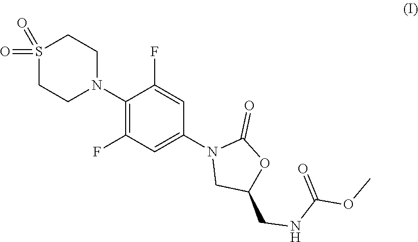 [SMILES: COC(=O)NC[C@H]1CN(c2cc(F)c(N3CCS(=O)(=O)CC3)c(F)c2)C(=O)O1]
or a pharmaceutically acceptable salt thereof, comprising treating a compound of Formula 5b 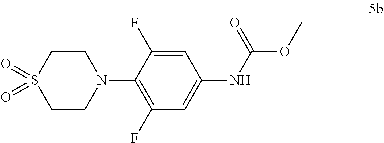 [SMILES: COC(=O)Nc1cc(F)c(N2CCS(=O)(=O)CC2)c(F)c1]
with methyl (S)-(3-chloro-2-hydroxypropyl)carbamate.

6. The method of claim 1 wherein the process of preparing a compound of Formula I 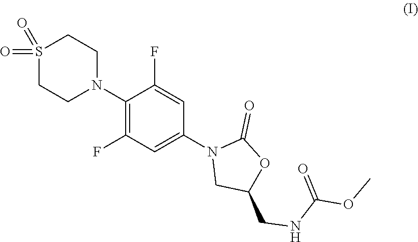 [SMILES: COC(=O)NC[C@H]1CN(c2cc(F)c(N3CCS(=O)(=O)CC3)c(F)c2)C(=O)O1]
or a pharmaceutically acceptable salt thereof, comprises treating a compound of Formula 5b 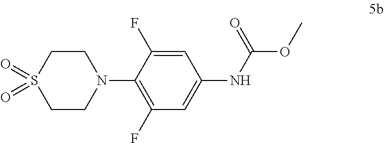 [SMILES: COC(=O)Nc1cc(F)c(N2CCS(=O)(=O)CC2)c(F)c1]
with methyl (S)-(3-chloro-2-hydroxypropyl)carbamate in the presence of a base.

7. The method of claim 6, wherein the base is selected from the group consisting of sodium methoxide, potassium methoxide, lithium methoxide sodium t-butoxide, potassium t-butoxide and lithium t-butoxide.

8. The method of claim 6, wherein the base is lithium t-butoxide.

9. A compound selected from:

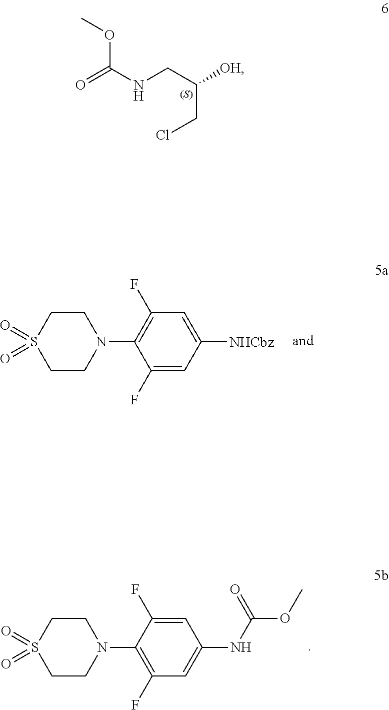

10. (canceled)

11. (canceled)

12. A crystalline form of the compound of Formula I:

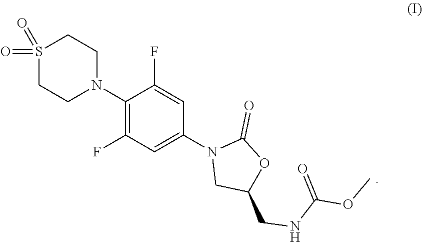 [SMILES: COC(=O)NC[C@H]1CN(c2cc(F)c(N3CCS(=O)(=O)CC3)c(F)c2)C(=O)O1]

13. The crystalline form of claim 12 characterized by an X-ray powder diffraction having at least four of the following 2θ values measured using Cu Kα of 4.49±0.2 2θ, 9.00±0.2 2θ, 10.55±0.2 2θ, 10.98±0.2 2θ, 13.00±0.2 2θ, 13.65±0.2 2θ, 16.31±0.2 2θ, 17.14±0.2 2θ, 17.50±0.2 2θ, 18.10±0.2 2θ, 19.64±0.2 2θ, 20.14±0.2 2θ, 21.03±0.2 2θ, 21.32±0.2 2θ, 21.90±0.2 2θ, 22.68±0.2 2θ, 24.55±0.2 2θ, 25.29±0.2 2θ and 26.00±0.2 2θ.

14. The crystalline form of claim 13 characterized by an X-ray powder diffraction having at the following 2θ values measured using Cu Kα: 10.55±0.2 2θ, and 13.65±0.2 2θ.

15. The crystalline form of claim 12 further characterized by the differential scanning calorimetric (DSC) curve of FIG. 3, or further characterized by the thermogravimetric analysis (TGA) curve of FIG. 2.

16. (canceled)

17. The crystalline form of claim 12 characterized by an X-ray powder diffraction having at least four of the following 2θ values measured using Cu Kα 4.53±0.2 2θ, 10.81±0.2 2θ, 13.35±0.2 2θ, 14.97±0.2 2θ, 16.76±0.2 2θ, 17.62±0.2 2θ, 18.14±0.2 2θ, 18.65±0.2 2θ, 18.88±0.2 2θ, 19.70±0.2 2θ, 20.62±0.2 2θ, 21.47±0.2 2θ, 22.00±0.2 2θ, 22.79±0.2 2θ, 23.76±0.2 2θ, 23.97±0.2 2θ, 25.42±0.2 2θ, 25.79±0.2 2θ, 26.30±0.2 2θ, 27.81±0.2 2θ, 28.86±0.2 2θ, and 30.05±0.2 2θ.

18. The crystalline form of claim 17 characterized by an X-ray powder diffraction having at least four of the following 2θ values measured using Cu Kα 13.35±0.2 2θ, 14.97±0.2 2θ, 20.62±0.2 2θ, 28.86±0.2 2θ and 30.05±0.2 2θ.

19. The crystalline form of claim 17 further characterized by the differential scanning calorimetric (DSC) curve of FIG. 6, or further characterized by the thermogravimetric analysis (TGA) curve of FIG. 5.

20. (canceled)

21. The crystalline form of claim 12 characterized by an X-ray powder diffraction having at least four of the following 2θ values measured using Cu Kα: consisting of 6.19±0.2 2θ, 8.15±0.2 2θ, 9.41±0.2 2θ, 10.19±0.2 2θ, 12.44±0.2 2θ, 13.12±0.2 2θ, 14.68±0.2 2θ, 15.44±0.2 2θ, 16.28±0.2 2θ, 18.69±0.2 2θ, 19.22±0.2 2θ, 20.21±0.2 2θ, 21.06±0.2 2θ, 21.65±0.2 2θ, 22.49±0.2 2θ, 22.84±0.2 2θ, 23.34±0.2 2θ, 23.93±0.2 2θ, 24.46±0.2 2θ and 24.99±0.2 2θ.

22. The crystalline form of claim 21 characterized by an X-ray powder diffraction having at least four of the following 2θ values measured using Cu Kα 6.19±0.2 2θ, 8.15±0.2 2θ, 9.41±0.2 2θ, 10.19±0.2 2θ, 12.44±0.2 2θ, 14.68±0.2 2θ, 19.22±0.2 2θ and 24.99±0.2 2θ.

23. The crystalline form of claim 21 further characterized by the differential scanning calorimetric (DSC) curve of FIG. 9 or further characterized by the thermogravimetric analysis (TGA) curve of FIG. 8.

24. The crystalline form of claim 12 characterized by an X-ray powder diffraction having at least four of the following 2θ values measured using Cu Kα: 5.72±0.2 2θ, 8.99±0.2 2θ, 11.44±0.2 2θ, 12.19±0.2 2θ, 15.63±0.2 2θ, 16.36±0.2 2θ, 16.56±0.2 2θ, 16.83±0.2 2θ, 17.05±0.2 2θ, 17.19±0.2 2θ, 18.07±0.2 2θ, 18.46±0.2 2θ, 19.52±0.2 2θ, 20.40±0.2 2θ, 21.15±0.2 2θ, 21.82±0.2 2θ, 23.40±0.2 2θ, 23.83±0.2 2θ, 24.14±0.2 2θ, 24.49±0.2 2θ, 26.10±0.2 2θ and 28.02±0.2 2θ.

25. The crystalline form of claim 24 characterized by an X-ray powder diffraction having the following 2θ values measured using Cu Kα5.72±0.2 2θ, 11.44±0.2 2θ, and 12.19±0.2 2θ.

26. The crystalline form of claim 24 further characterized by the differential scanning calorimetric (DSC) curve of FIG. 12 or further characterized by the thermogravimetric analysis (TGA) curve of FIG. 11.

27. A method of treating a mycobacterial infection caused by Mycobacteria tuberculosis comprising administering to a patient in need of such treatment a therapeutically effective amount of a compound of claim 12.

28. (canceled)

29. A pharmaceutical composition comprising a drug substance that comprises crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate of claim 12 and a pharmaceutically acceptable carrier.

30. The pharmaceutical composition of claim 29, wherein at least 50% by weight of the crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate is present in the drug substance.

31. A pharmaceutical composition of claim 29, wherein at least 5% by weight of the crystalline methyl ({(5S)-3-[4-(1,1-dioxo-1λ⁶-thiomorpholin-4-yl)-3,5-difluorophenyl]-2-oxo-1,3-oxazolidin-5-yl}methyl)carbamate is present in the drug substance.

32. An amorphous dispersion formulation of the compound of Formula I 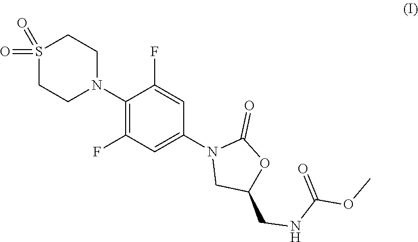 [SMILES: COC(=O)NC[C@H]1CN(c2cc(F)c(N3CCS(=O)(=O)CC3)c(F)c2)C(=O)O1]
comprising an amorphous form of the compound of Formula I and a polymer.

33. The amorphous dispersion formulation of claim 32, wherein the polymer is hydroxypropyl methyl cellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone or polyvinylpyrrolidinone-polyvinylacetate copolymers.

34. The amorphous dispersion formulation of claim 32, wherein the polymer is hydroxypropyl methyl cellulose acetate succinate or polyvinylpyrrolidinone-polyvinylacetate copolymer.

35. (canceled)

36. The amorphous dispersion formulation of claim 32, wherein the amorphous dispersion formulation is made by hot melt extrusion.
